SEQUENCE LISTING (3 sequences)

SEQ ID NO: 1 - protein, 27 aa, Artificial Sequence
  APTX (C2H2)
PLRCHECQQLLPSIPQLKEHLRKHWTQ

SEQ ID NO: 2 - protein, 29 aa, Artificial Sequence
  PARP-1 ZF 1 (C3H1)
GRASCKKCSESIPKDKVPHWYHFSCFWKV

SEQ ID NO: 3 - protein, 37 aa, Artificial Sequence
  XPA (C4)
DYVICEECGKEFMDSYLMNHFDLPTCDNCRDADDKHK

(19) United States
(12) Patent Application Publication
(10) Pub. No.: US 2016/0184356 A1
(43) Pub. Date: Jun. 30, 2016

(54) ARSENIC-BASED TREATMENT OF CANCERS AND INFLAMMATORY DISORDERS

(71) Applicants: Ke Jian Jim LIU, (US); Laurie HUDSON, (US); Karen COOPER, (US); Xixi ZHOU, (US); STC.UNM, Albuquerque, NM (US)

(72) Inventors: Ke Jian Jim Liu, Albuquerque, NM (US); Laurie Hudson, Albuquerque, NM (US); Karen Cooper, Tijerar, NM (US); Xixi Zhou, Albuquerque, NM (US)

(21) Appl. No.: 14/776,929

(22) PCT Filed: Mar. 14, 2014
(86) PCT No.: PCT/US14/27218

§ 371 Date: Sep. 15, 2015

Related U.S. Application Data
(60) Provisional application No. 61/788,537, filed on Mar. 15, 2013.

(51) Int. Cl. A61K 33/36; A61K 45/06; A61K 41/00

(52) U.S. Cl. CPC A61K 33/36; A61K 41/0038; A61K 45/06; A61N 2005/1098

(57) ABSTRACT

In one embodiment, the invention provides a method of treating a subject who suffers from a cancer (particularly a chemotherapeutic or radiotherapeutic-resistant cancer) or from an inflammatory disorder, the method comprising co-administering to the subject a pharmaceutically-effective amount of: (a) one or more elements or compounds selected from the group consisting of arsenic, an arsenite and arsenic trioxide (ATO); and optionally (b) at least one anticancer agent (preferably a DNA damaging agent); and/or (c) at least one or more Poly(ADP-ribose) polymerase (PARP) inhibitors other than arsenic, an arsenite and arsenic trioxide (ATO). In preferred embodiments, the arsenic for the treatment of cancer, arsenite and ATO serve as radio sensitizers for concomitant radiotherapy as well as PARP inhibitors. Methods for treating inflammatory disease are also disclosed. Related pharmaceutical formulations are also provided.

### CROSS REFERENCE TO RELATED APPLICATIONS

This application claims priority from U.S. Provisional Patent Application No. 61/788,537, entitled “Arsenic Trioxide as an Inhibitor of PARP-1 and Treatments of Disease Modulated Through Same”, filed Mar. 15, 2013. The complete contents of this provisional application are hereby incorporated herein in their entirety.

### STATEMENT REGARDING FEDERAL FUNDING

This invention was made with government support under National Institute of Health Grant Nos. R01ES15826 and R01ES021100. Consequently, the United States has certain rights in the invention.

### FIELD OF THE INVENTION

The present invention relates to the unexpected discovery that arsenic, an arsenite and arsenic trioxide (ATO), in particular ATP, exhibit activity as Poly(ADP-ribose) polymerase 1 (PARP-1) inhibitors. Preferably, ATO is used as the PARP-1 inhibitor in the present invention.

Accordingly, in one embodiment, the invention provides a method of treating a subject who suffers from a cancer (in certain embodiments, a chemotherapeutic or radiotherapeutic-resistant cancer which is recurrent) or from an inflammatory disorder, the method comprising administering to the subject an effective amount of a compound selected from the group consisting of arsenic, an arsenite, arsenic trioxide (ATO) and mixtures thereof as PARP-1 inhibitors. Preferably, ATO is the PARP-1 inhibitor compound used to effect therapy in the subject.

In other embodiments, the invention provides a method of treating a subject who suffers from cancer, the method comprising administering to the subject an effective amount of at least one anticancer compound (preferably, a DNA damaging chemotherapeutic agent) alone or in combination with radiation therapy, further in combination with arsenic, an arsenite, arsenic trioxide (ATO) or mixtures thereof as a PARP-1 inhibitor. Preferably, arsenic trioxide (ATO) is the agent used in combination with the anticancer compound and/or the radiation therapy.

In still other embodiments, the invention provides a method of treating a subject who suffers from cancer, the method comprising administering an effective amount of arsenic, an arsenite, arsenic trioxide (ATO) or mixtures thereof as a PARP-1 inhibitor in combination with radiation therapy. In this method, at least one additional anticancer agent may be used. Optionally, another PARP inhibitor (often a PARP 1 or PARP 2 inhibitor) may also be used for its additive or synergistic activity when combined with arsenic, an arsenite, arsenic trioxide (ATO) or mixtures thereof.

It has been discovered unexpectedly by the inventors of the present application that arsenic, an arsenite, arsenic trioxide (ATO) or mixtures thereof (often, ATO in the absence of the other arsenic compounds) exhibit PARP-1 inhibitory activity and may be favorably used therapeutically to treat cancer and inflammatory diseases as otherwise disclosed herein. In particular aspects of the invention, ATO is used to treat cancer, optionally (preferably) in combination with a chemotherapeutic agent (often, a DNA damaging agent) and/or radiation therapy, optionally in further combination with an additional PARP inhibitor. This approach to cancer therapy results in the treatment of cancer which inhibits/causes death of cancer cells (often to a greater degree than an anticancer agent and another PARP inhibitor) and prevents treated cancer cells from repairing the DNA which is damaged by the chemotherapeutic agent and/or the radiation therapy. The result is a more effective approach to cancer therapy. This treatment approach results in inhibition of cancer cell growth and/or death of cancer cells and inhibits the growth of chemotherapy-resistant and/or radiation resistant cancer cells which often occurs after an initial treatment of chemotherapy and/or radiation therapy. A further result of the present invention is that recurrent cancers occur less often and when they do, the recurrent cancers are less likely to be chemotherapy-resistant and/or radiation resistant, thus allowing any recurrent cancer to be treated more easily with conventional therapies (chemotherapy and/or radiation).

### BACKGROUND OF THE INVENTION

Arsenic exists in inorganic and organic forms. Arsenite is an inorganic trivalent arsenic compound widely presents in water, soil, and food.¹ In contrast, arsenic trioxide (ATO, As₂O₃), another trivalent arsenic compound, is the most common inorganic arsenical in airborne dust.¹ Organic arsenicals mainly consist of mono- and di-methylated arsenic metabolites, derived from biomethylation of inorganic arsenicals in cellular environment.⁸⁻¹¹ A trivalent mono-methylated arsenic metabolite, monomethylarsonous acid (MMA(III)), has been shown to display greater toxicity and/or carcinogenic potential than inorganic arsenite.¹²⁻¹⁴

Interaction with zinc finger proteins is considered to be an important mechanism of arsenic toxicity and carcinogenesis. Substitution of zinc with another metal, such as arsenic, is believed to disrupt the coordination sphere in the finger environment and consequently the zinc finger function.^{1,15} Furthermore, both inorganic and organic trivalent arsenic compounds interact with zinc finger proteins. Zinc finger proteins, poly (ADP-ribose) polymerase 1 (PARP-1) and xeroderma pigmentosum group A (XPA) are both involved in DNA repair and have been validated as direct molecular targets for arsenite and MMA(III).^{1-5,16-18} ATO is used therapeutically to treat acute prornyelocytic leukemia (APL), a hematological cancer caused by the PML/RARA oncogene. ATO targets PML/RARA for degradation thereby acting as a very specific agent for the treatment of APL and effectively cures most APL patients. ATO binds to the PML/RARA zinc finger¹⁹ See, Breccia M, Lo-Coco F. Arsenic trioxide for management of acute promyelocytic leukemia: current evidence on its role in front-line therapy and recurrent disease. Expert Opin Pharmacother. 2012 May; 13(7):1031-43. doi: 10.1517/14656566.2012.677436. Epub 2012 Apr. 3. Review. PubMed PMID: 22468778. Also, Lallemand-Breitenbach V, Zhu J, Chen Z, de Thé H. Curing APL through PML/RARA degradation by As2O3. Trends Mol Med. 2012 January; 18(1):36-42. doi: 10.1016/j.molmed.2011.10.001. Epub 2011 Nov. 4. Review. PubMed PMID: 22056243.

We have investigated extensively the interaction of arsenite with zinc finger proteins in recent years. Our findings demonstrate that arsenite can replace zinc in the zinc finger moiety, leading to changes of structure and loss of protein function.²⁰ In addition, we found that arsenite selectively interacts with zinc finger motifs with C3H1 or C4 configurations by coordinating with three cysteine residues.^{1,21} This suggests that subsets of zinc finger proteins are more sensitive molecular targets of arsenite than others. Since a methyl group already occupies one of the three covalent bonds in MMA(III), it is likely that MMA(III) will not be able to bind with three cysteine residues as arsenite does. Therefore, we hypothesize that binding selectivity of MMA(III) will be different from that of arsenite.

### SUMMARY OF THE INVENTION

The inventors tested the differential binding selectivity hypothesis by investigating interactions of arsenite, ATO, and MMA(III) with three different configurations of zinc finger peptides and proteins: C2H2 (aprataxin, APTX), C3H1 (PARP-1), and C4 (XPA). A variety of analytical approaches were utilized to determine whether the three arsenicals display differential binding selectivity toward these zinc finger configurations, and potential consequences of these interactions in terms of structural or functional changes. The results demonstrate that the binding selectivity indeed differs among the methylated versus non-methylated arsenicals, which provides insightful understanding for the molecular mechanisms underlying the differential effects of inorganic versus organic arsenicals in arsenic toxicity and carcinogenesis. From these studies we have determined that arsenic, an arsenite and arsenic trioxide (ATO) are PARP-1 inhibitors and that ATO is particularly useful in the treatment of cancer and inflammatory diseases because of this unexpectedly discovered activity.

The present invention provides the bases for novel and clinically-significant therapies that supplement and complement known anti-cancer and anti-inflammatory regimens.

In a first embodiment, the present invention is directed to a method of treating cancer in a patient or subject in need comprising co-administering to the patient subject a pharmaceutically effective amount of:

(a) one or more elements or compounds selected from the group consisting of arsenic, an arsenite and arsenic trioxide (ATO), preferably ATO; and
(b) at least one anticancer agent (often, a DNA damaging chemotherapeutic agent),
wherein the administration of said arsenic, arsenite and mixtures thereof and said anticancer agent is optionally (and preferably) combined with radiation therapy of said cancer and/or an additional PARP inhibitor.

In an additional embodiment, the present invention is directed to a method of treating cancer in a patient or subject in need comprising administering a pharmaceutically effective amount of:

(a) one or more elements or compounds selected from the group consisting of arsenic, an arsenite and arsenic trioxide (ATO), preferably ATO, in combination with radiation therapy,
(b) optionally (preferably), at least one anticancer agent (often, a DNA damaging chemotherapy agent), and
(c) further optionally, an additional PARP inhibitor.

As disclosed above, in certain embodiments according to the present invention, arsenic, an arsenite, and/or ATO are administered to the cancer patient with at least one additional PARP inhibitor, the resulting combination of PARP inhibitors (which includes arsenic, arsenite and/or ATO (preferably, ATO in the absence of arsenic and/or arsenite) often providing a synergistic effect in the treatment of cancer.

In certain preferred embodiments, the arsenic, arsenite and/or ATO serve as radiosensitizers for concomitant radiotherapy. In certain embodiments, ATO is administered in effective amounts alone or in combination with an effective amount of an additional PARP inhibitor as otherwise described herein for the treatment of cancer, which treatment method may be optionally combined with chemotherapy (preferably at least one DNA damaging agent) and/or radiation therapy.

Related pharmaceutical formulations pursuant to the present invention are also provided.

In a particular embodiment, the present invention provides a method of treating a subject who suffers from a cancer selected from the group consisting of breast cancer, ovarian cancer, colorectal cancer, glioblastoma multiform (GBM), melanoma, lung cancer and a glioma, the method comprising co-administering to the subject a pharmaceutically-effective amount of:

(a) one or more elements or compounds selected from the group consisting of arsenic, an arsenite and ATO (preferably ATO);
(b) one or more anticancer agents (often, a DNA damaging chemotherapeutic agent),
wherein the administration of said arsenic, arsenite and mixtures thereof and said anticancer agent is optionally (and preferably) combined with radiation therapy of said cancer and/or an additional PARP inhibitor. In certain embodiments, the arsenic, arsenite and/or ATO are administered in combination with an additional PARP inhibitor in the treatment of these cancers.

“PARP inhibitors” include PARP-1 inhibitors and PARP-2 inhibitors.

In a preferred embodiment, the subject is treated concomitantly by radiotherapy and the one or more elements or compounds selected from the group consisting of arsenic, an arsenite and ATO are administered to the subject as a radiosensitizer prior to or during radiotherapy.

As described, in certain embodiments, the subject is also treated concomitantly by chemotherapeutic agents which are DNA damaging agents, preferably including such agents as paclitaxel and docetaxel, platinum-based antineoplastics (e.g. cisplatin, carboplatin, oxaliplatin, satraplatin, picoplatin, Nedaplatin, Triplatin, and Lipoplatin). In certain additional embodiments further treatment of a cancer using hormonal therapy, proton therapy, cryosurgery, and/or high intensity focused ultrasound (HIFU) is used, depending upon clinical assessments and treatment goals.

In certain embodiments, the subject suffers from a treatment-resistant cancer, e.g. breast cancer in which Breast Cancer Type 1 Susceptibility Protein (BRCA1)-deficient cells exhibit decreased sensitivity to PARP inhibitors; hormone and castration-resistant prostate cancer; metastatic melanoma; drug resistant childhood acute lymphoblastic leukemia (ALL); and chemotherapy and radiotherapy-resistant non-small cell lung cancer, glioblastomas, cervical cancer, esophageal cancer (EC), breast cancers and non-small cell lung cancer.

In a further embodiment, the invention provides a method of treating a subject who suffers from cancer wherein the cancer has developed resistance to a PARP inhibitor, the method comprising administering an effective amount of

(a) one or more elements or compounds selected from the group consisting of arsenic, an arsenite and ATO; and optionally
(b) one or more additional PARP inhibitors to which the cells have not become resistant; and/or
(c) at least one anticancer agent (preferably, a DNA damaging agent), wherein the method may be combined with radiation therapy.

In still another embodiment, invention provides a method of treating a subject who suffers an inflammatory disorder, the method comprising co-administering to the subject a pharmaceutically-effective amount of:

(a) one or more elements or compounds selected from the group consisting of arsenic, an arsenite and ATO; and optionally
(b) one or more PARP inhibitors and/or an additional anti-inflammatory agent.

Pharmaceutical formulations that are useful in the treatment of a variety of cancers and inflammatory disorders are also provided. These formulations comprise (a) one or more elements or compounds selected from the group consisting of arsenic, an arsenite and ATO;

(b) optionally, one or more PARP inhibitors (preferably, a PARP-1 inhibitor);
(c) further optionally, one or more additional anti-cancer agents (e.g. platinum-based anti-neoplastics); and
(d) optionally, a pharmaceutically-acceptable excipient.

In certain embodiments, by combining synergistically PARP-inhibiting active ingredients and PARP-targeting/radiotherapy-sensitizing arsenic, arsenite and ATO, the methods and formulations described herein prove particularly effective in treating a wide variety of cancers that have been previously been associated with high rates of remission and poor long-term survival, especially when combined with a chemotherapy agent (preferably, a DNA damaging agent) and/or radiation therapy.

These and other aspects of the invention are described further in the Detailed Description of the Invention.

### BRIEF DESCRIPTION OF THE FIGURES

FIG. 1. Cobalt spectrometry analysis for arsenicals binding with zinc fingers. APTX, PARP-1 and XPA zinc finger peptides were pre-incubated with arsenic compounds for 30 min. After that, 200 μM cobalt were added into the system. Absorbance at 660 nm increased when cobalt bound to zinc finger motifs. A, Cobalt binding signal decreased in PARP-1 and XPA zinc fingers after pre-incubating with increasing concentrations of arsenite. For the APTX zinc finger, free metal binding site was always available. B, cobalt binding signal decreased in all three configurations of zinc fingers in a MMA(III) concentration dependent manner. C, ATO performed the same in cobalt spectrum detection as arsenite. Data were presented as mean±SD, * p<0.05 vs. corresponding [As]=0 group, n=3.

FIG. 2. As—S bound formation analysis using UV-Vis spectrometry. Arsenic compounds (A, arsenite, B, MMA(III), C, ATO) were incubated with 100 μM of the indicated zinc finger peptides at room temperature for 30 min. Then UV-Vis spectrometry analysis was performed as described in the methods section. Absorbance at 270 nm represents As—S bound formation after arsenic binds to zinc fingers. A, arsenite bound to C3 and C4 zinc fingers selectively. B, MMA(III) bound to all three configurations of zinc fingers. C, ATO showed the same zinc finger binding selectivity as arsenite. Data were presented as mean±SD, * p<0.05 vs. corresponding [As]=0 group, n=3.

FIG. 3. Zinc finger binding behaviors analyzed by MALDI-TOF mass spectrometry. 100 μM arsenic compounds were incubated with 100 μM of the indicated zinc finger peptides at room temperature for 30 min, then MALDI-TOF mass spectrometry analysis was performed as described in methods section. A, B, and C, apo-APTX, PARP-1 and XPA zinc fingers had m/z at 3319, 3454 and 4400 in mass spectra. D, arsenite did not bind to C2H2 zinc finger (APTX). E and F, arsenite bound to C3H1 (PARP-1) and C4 (XPA) zinc fingers, giving a +72 m/z shift. G, H, and I, MMA(III) could bind to all three configurations of zinc fingers, giving +88 m/z shift to zinc fingers. In I, 1 or 2 molecules of MMA(III) bound to C4 zinc finger (XPA). Each molecules of MMA(III) gave +88 m/z shift. J, K, and L, ATO showed the same zinc finger binding selectivity as arsenite. ATO and arsenite gave the same +72 m/z shift to C3H1 and C4 zinc fingers.

FIG. 4. Conformational changes of zinc fingers induced by arsenic binding. Intrinsic fluorescence analysis was performed as described in the methods section. Intensities of fluorescence at 350 nm were used to represent the conformation/folding status of zinc finger peptides. The fluorescent intensities of 100 μM zinc treatment on zinc finger peptides are shown (top left corner) as controls. A, Natural conformation of APTX could be altered by MMA(III) in a concentration dependent manner, while arsenic and ATO showed no effect. B, all three arsenic compounds could cause conformational change on PARP-1 zinc finger in a concentration dependent manner. C, all three arsenic compounds could cause conformational change on XPA zinc finger in a concentration dependent manner. Data were presented as mean±SD, * p<0.05 vs. corresponding [As]=0 group, n=3.

FIG. 5. Zinc loss from zinc finger proteins isolated from cells treated with arsenicals. HaCat cells were treated with 2 μM of different arsenic compounds for 24 h. Zinc finger proteins were immunoprecipitated from cell extract, then zinc content in each specific protein were analyzed with colorimetric assay as described in the methods section. Zinc content in APTX protein was sensitive only to MMA(III) treatment, but zinc in PARP-1 and XPA was decreased by all three arsenic compounds. i. e, MMA(III) could remove zinc from all three configurations of zinc finger proteins, while arsenic and ATO selectively remove zinc from C3H1 and C4 zinc finger proteins in cells. Bar plot shows mean±SD, * p<0.05 vs. Ctrl group (no treatment), n=3.

FIG. 6. Schematic illustration of arsenite, ATO and MMA(III) binding to zinc fingers. Arsenite and ATO bind to zinc fingers by coordinating with 3 Cys, which leads to selective binding with C3H1 and C4 zinc fingers. MMA(III) binds to zinc fingers together with methyl group (-Me), using 2 Cys instead of 3, which causes nonselective binding with all 3 configurations of zinc fingers.

FIG. 7. Ovarian cancer cells (SKOV3ip) were treated for 48 hours with the following as indicated 3 μM AG-014699 (PARO inhibitor), 1 μM cisplatin (CP), 1 μM arsenic trioxide (ATO) or combination thereof for 48 hours, treatments were removed and viable cells were allowed to grow for 48 hours. *p<0.05. The findings demonstrate that the combination of arsenic trioxide and cisplatin causes greater loss of cell viability than either agent alone and that this combination is more effective than combining cisplatin with a conventional PARP inhibitor AG-014699.

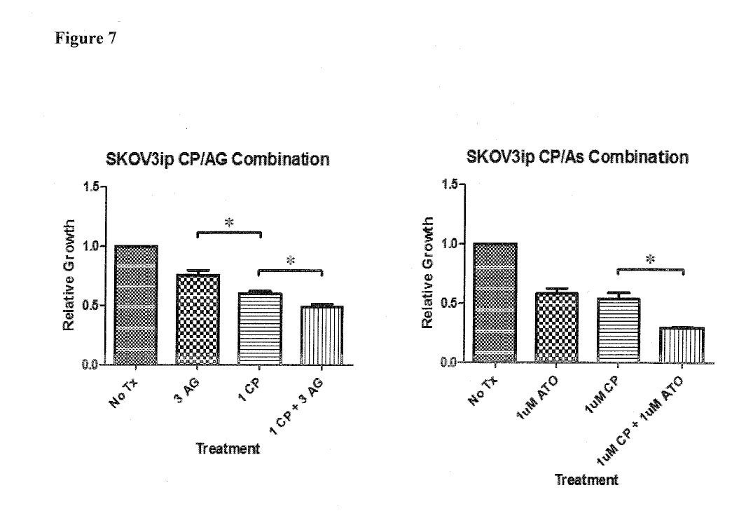

### DETAILED DESCRIPTION OF THE INVENTION

The following terms are used throughout the specification to describe the present invention. Where a term is not given a specific definition herein, that term is to be given the same meaning as understood by those of ordinary skill in the art. The definitions given to the disease states or conditions which may be treated using one or more of the compounds according to the present invention are those which are generally known in the art.

It is noted that, as used in this specification and the appended claims, the singular forms “a,” “an,” and “the,” include plural referents unless expressly and unequivocally limited to one referent. Thus, for example, reference to “a compound” includes two or more different compound. As used herein, the term “include” and its grammatical variants are intended to be non-limiting, such that recitation of items in a list is not to the exclusion of other like items that can be substituted or other items that can be added to the listed items.

Arsenites include, but are not limited to, arsenites selected from the group consisting of AsO₃³⁻ (ortho-arsenite); [AsO₂⁻]ₙ (meta-arsenite); As₂O₅⁴⁻ (pyro-arsenite); As₃O₇⁵⁻ (a polyarsenite, [O₂As—O—As(O)—O—AsO₂]); As₄O₉⁶⁻ (a polyarsenite, [O₂As—O—As(O)—O—As(O)—O—AsO₂]); and [As₆O₁₁⁴⁻]ₙ, (a polymeric anion).

The term “patient” or “subject” is used throughout the specification to describe an animal, preferably a human, to whom treatment, including prophylactic treatment, with the compositions according to the present invention is provided (a patient or subject in need). For treatment of those infections, conditions or disease states which are specific for a specific animal such as a human patient, the term patient refers to that specific animal. In many instances, diagnostic methods are applied to patients or subjects who are suspected of having cancer or a inflammatory disorder or who have cancer or a inflammatory disorder and the diagnostic method is used to assess the severity of the disease state or disorder.

The term “compound” is used herein to refer to any specific chemical compound disclosed herein and in particular, arsenic trioxide (ATO). Within its use in context, the term generally refers to a single small molecule as disclosed herein, but in certain instances may also refer to other forms of the compound. The term compound includes active metabolites of compounds and/or pharmaceutically acceptable salts thereof.

The term “effective amount” is used throughout the specification to describe concentrations or amounts of formulations or other components which are used in amounts, within the context of their use, to produce an intended effect according to the present invention, for example to damage DNA as a chemotherapy agent or by exposure to radiation, to inhibit PARP (e.g. PARP-1) and treat disease states and/or conditions which are modulated through PARP (e.g. PARP-1). The formulations or component(s) may be used to produce a favorable change in a disease or condition treated, whether that change is a remission of effects of a disease state or condition, a favorable physiological result, a reversal or attenuation of a disease state or condition treated, the prevention or the reduction in the likelihood of a condition or disease-state occurring, depending upon the disease or condition treated. Where formulations are used in combination, each of the formulations is used in an effective amount, wherein an effective amount may include a synergistic amount. The amount of formulation used in the present invention may vary according to the nature of the formulation, the age and weight of the patient and numerous other factors which may influence the bioavailability and pharmacokinetics of the formulation, the amount of formulation which is administered to a patient generally ranges from about 0.001 mg/kg to about 50 mg/kg or more, about 0.5 mg/kg to about 25 mg/kg, about 0.1 to about 15 mg/kg, about 1 mg to about 10 mg/kg per day and otherwise described herein. The person of ordinary skill may easily recognize variations in dosage schedules or amounts to be made during the course of therapy.

The term “prophylactic” is used to describe the use of a formulation described herein which reduces the likelihood of an occurrence of a condition or disease state in a patient or subject. The term “reducing the likelihood” refers to the fact that in a given population of patients, the present invention may be used to reduce the likelihood of an occurrence, recurrence or metastasis of disease in one or more patients within that population of all patients, rather than prevent, in all patients, the occurrence, recurrence or metastasis of a disease state.

The term “pharmaceutically acceptable” refers to a salt form or other derivative (such as an active metabolite or prodrug form) of the present compounds or a carrier, additive or excipient which is not unacceptably toxic to the subject to which it is administered.

“Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc. Treatment, as used herein, encompasses both prophylactic and therapeutic treatment.

An “inflammatory disorder” includes, but is not limited to, lung diseases, hyperglycemic disorders including diabetes and disorders resulting from insulin resistance, such as Type I and Type II diabetes, as well as severe insulin resistance, hyperinsulinemia, and dyslipidemia (e.g. hyperlipidemia (e.g., as expressed by obese subjects), elevated low-density lipoprotein (LDL), depressed high-density lipoprotein (HDL), and elevated triglycerides) and insulin-resistant diabetes, such as Mendenhall's Syndrome, Werner Syndrome, leprechaunism, and lipoatrophic diabetes, renal disorders, such as acute and chronic renal insufficiency, end-stage chronic renal failure, glomerulonephritis, interstitial nephritis, pyelonephritis, glomerulosclerosis, e.g., Kimmelstiel-Wilson in diabetic patients and kidney failure after kidney transplantation, obesity, GH-deficiency, GH resistance, Turner's syndrome, Laron's syndrome, short stature, increased fat mass-to-lean ratios, immunodeficiencies including decreased CD4⁺ T cell counts and decreased immune tolerance or chemotherapy-induced tissue damage, bone marrow transplantation, diseases or insufficiencies of cardiac structure or function such as heart dysfunctions and congestive heart failure, neuronal, neurological, or neuromuscular disorders, e.g., diseases of the central nervous system including Alzheimer's disease, or Parkinson's disease or multiple sclerosis, and diseases of the peripheral nervous system and musculature including peripheral neuropathy, muscular dystrophy, or myotonic dystrophy, and catabolic states, including those associated with wasting caused by any condition, including, e.g., mental health condition (e.g., anorexia nervosa), trauma or wounding or infection such as with a bacterium or human virus such as HIV, wounds, skin disorders, gut structure and function that need restoration, and so forth.

“Inflammatory disorder” also includes a cancer and an “infectious disease” as defined herein, as well as disorders of bone or cartilage growth in children, including short stature, and in children and adults disorders of cartilage and bone in children and adults, including arthritis and osteoporosis. An “inflammation-associated metabolic disorder” includes a combination of two or more of the above disorders (e.g., osteoporosis that is a sequela of a catabolic state). Specific disorders of particular interest targeted for treatment herein are diabetes and obesity, heart dysfunctions, kidney disorders, neurological disorders, bone disorders, whole body growth disorders, and immunological disorders.

In one embodiment, an “inflammatory disorder” includes central obesity, dyslipidemia including particularly hypertriglyceridemia, low HDL cholesterol, small dense LDL particles and postpranial lipemia; glucose intolerance such as impaired fasting glucose; insulin resistance and hypertension, and diabetes. The term “diabetes” is used to describe diabetes mellitus type I or type II. The present invention relates to a method for improving renal function and symptoms, conditions and disease states which occur secondary to impaired renal function in patients or subjects with diabetes as otherwise described herein. It is noted that in diabetes mellitus type I and II, renal function is impaired from collagen deposits, and not from cysts in the other disease states treated by the present invention.

A “neurodegenerative disorder” or “neuroinflammation” is encompassed by the definition of “inflammatory disorder” and includes, but is not limited to, Alzheimer's Dementia (AD), amyotrophic lateral sclerosis, depression, epilepsy, Huntington's Disease, multiple sclerosis, the neurological complications of AIDS, spinal cord injury, glaucoma and Parkinson's disease.

The term “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Cancers generally show partial or complete lack of structural organization and functional coordination with the normal tissue and most invade surrounding tissues, metastasize to several sites, and are likely to recur after attempted removal and to cause the death of the patient unless adequately treated. As used herein, the term cancer is used to describe all cancerous disease states applicable to treatment according to the present invention and embraces or encompasses the pathological process associated with all virtually all epithelial cancers, including carcinomas, malignant hematogenous, ascitic and solid tumors. Examples of cancers which may be treated using methods according to the present invention include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas. See, for example, The Merck Manual of Diagnosis and Therapy, 17.sup.th ed. (Whitehouse Station, N.J.: Merck Research Laboratories, 1999) 973-74, 976, 986, 988, 991).

In addition to the treatment of ectopic cancers as described above, the present invention also may be used preferably to treat eutopic cancers such as choriocarcinoma, testicular choriocarcinoma, non-seminomatous germ cell testicular cancer, placental cancer (trophoblastic tumor) and embryonal cancer, among others.

An “immune disorder” is encompassed by the definition of “inflammatory disorder” and includes, but is not limited to, lupus, multiple sclerosis, rheumatoid arthritis, psoriasis, Type I diabetes, complications from organ transplants, xeno transplantation, diabetes, cancer, asthma, atopic dermatitis, autoimmune thyroid disorders, ulcerative colitis, Crohn's disease, Alzheimer's disease and leukemia.

The term “neoplasia” refers to the uncontrolled and progressive multiplication of tumor cells, under conditions that would not elicit, or would cause cessation of, multiplication of normal cells. Neoplasia results in a “neoplasm”, which is defined herein to mean any new and abnormal growth, particularly a new growth of tissue, in which the growth of cells is uncontrolled and progressive. Thus, neoplasia includes “cancer”, which herein refers to a proliferation of tumor cells having the unique trait of loss of normal controls, resulting in unregulated growth, lack of differentiation, local tissue invasion, and/or metastasis.

As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of anaplasia, or loss of differentiation and orientation of cells, and have the properties of invasion and metastasis. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17.sup.th ed. (Whitehouse Station, N.J.: Merck Research Laboratories, 1999) 973-74, 976, 986, 988, 991.

The term “anticancer agent” or “additional anticancer agent” shall mean chemotherapeutic agents (a chemotherapy agent, to the exclusion of an arsenic based compound or other PARP inhibitor) including such as an agent selected from the group consisting of microtubule-stabilizing agents, microtubule-disruptor agents, alkylating agents, antimetabolites, epidophyllotoxins, antineoplastic enzymes, topoisomerase inhibitors, inhibitors of cell cycle progression, and platinum coordination complexes. These may be selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)_{X} where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafamib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, among others.

The term “DNA damaging agent” refers to a chemotherapeutic agent which damages DNA of a cancer cell either directly or indirectly in its actions. Many chemotherapy agents are considered DNA damaging agents. Preferred agents include Alkylating agents, including nitrogen mustards: such as mechlorethamine (nitrogen mustard), chlorambucil, cyclophosphamide (Cytoxan®), ifosfamide, and melphalan; Nitrosoureas, including streptozocin, carmustine (BCNU), and lomustine; Alkyl sulfonates, including busulfan; Triazines, including dacarbazine (DTIC) and temozolomide (Temodar®); Ethylenimines, including thiotepa and altretamine (hexamethylmelamine); Platinum drugs, including cisplatin, carboplatin and oxalaplatin; Antimetabolites including fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, Thioguanine; Anti-tumor antibiotics including Anthracyclines, such as Daunorubicin, Doxorubicin (Adriamycin®), Epirubicin, Idarubicin and non-anthracycline antibioitics Actinomycin-D, Bleomycin and Mitomycin-C; Topoisomerase inhibitors including topotecan and irinotecan (CPT-11), etoposide (VP-16), teniposide and Mitoxantrone; Mitotic inhibitors, including Taxanes: paclitaxel (Taxol®) and docetaxel (Taxotere®); Epothilones, including ixabepilone (Ixempra®); Vinca alkaloids, including vinblastine (Velban®), vincristine (Oncovin®), and vinorelbine (Navelbine®), Estramustine (Emcyt®); and Targeted therapies including imatinib (Gleevec®), gefitinib (Iressa®), sunitinib (Sutent®) and bortezomib (Velcade®), among others.

The terms “radiotherapy” and “radiation therapy” are used interchangeably and describe therapy for cancer, especially including prostate cancer, which may be used in conjunction with the present compounds which exhibit activity as Intnase inhibitors having inherent anticancer activity. Radiation therapy uses high doses of radiation, such as X-rays, to destroy cancer cells. The radiation damages the genetic material of the cells so that they cannot grow. Although radiation damages normal cells as well as cancer cells, the normal cells can repair themselves and function, while the cancer cells cannot.

Radiation therapy may be used in combination with the presently claimed compounds, which inhibit Intnase and consequently, the cancer cells' ability to repair damage done by the radiation, thus potentiating radiation therapy. Radiation therapy is most effective in treating cancers that have not spread (metastasized). But it also may be used if the cancer has spread to nearby tissue. Radiation is sometimes used after surgery to destroy any remaining cancer cells and to relieve pain from metastatic cancer.

Radiation is delivered in one of two ways: External-beam radiation therapy and branchytherapy. External-beam radiation therapy uses a large machine to aim a beam of radiation at the tumor. After the area of cancer is identified, an ink tattoo no bigger than a pencil tip is placed on the skin of the subject so that the radiation beam can be aimed at the same spot for each treatment. This helps focus the beam on the cancer to protect nearby healthy tissue from the radiation. External radiation treatments usually are done 5 days a week for 4 to 8 weeks or more. If cancer has spread, shorter periods of treatment may be given to specific areas to relieve pain.

There are basically three types of external radiation therapy: conformal radiotherapy (3D-CRT), intensity-modulation radiation therapy (IMRT) and proton therapy. Conformal radiotherapy uses a three-dimensional planning system to target a strong dose of radiation to the cancer. This helps to protect healthy tissue from radiation. Intensity-modulated radiation therapy uses a carefully adjusted amount of radiation. This protects healthy tissues more than conformal radiotherapy does. Proton therapy uses a different type of energy (protons) than X-rays. This approach allows a higher amount of specifically directed radiation, which protects nearby healthy tissues the most. Sometimes proton therapy is combined with X-ray therapy.

Brachytherapy, or internal radiation therapy, uses dozens of tiny seeds that contain radioactive material. It may be used preferably to treat early-stage prostate and other cancer which is localized. Needles are used to insert the seeds through the skin into tissue, most often the prostate. The surgeon uses ultrasound to locate the tissue and guide the needles. As the needles are pulled out, the seeds are left in place. The seeds release radiation for weeks or months, after which they are no longer radioactive. The radiation in the seeds can't be aimed as accurately as external beams, but they are less likely to damage normal tissue. After the seeds have lost their radioactivity, they become harmless and can stay in place.

Radiation therapy may combine brachytherapy with low-dose external radiation. In other cases, treatment combines surgery with external radiation. In the present invention, compounds which are otherwise claimed may be used as radiation sensitizers to enhance or potentiate the effect of radiation by inhibiting the ability of the cancer tissue to repair the damage done by the radiation therapy.

Formulations of the invention may include a pharmaceutically acceptable diluent, carrier, solubilizer, emulsifier, preservative and/or adjuvant. Acceptable formulation materials preferably are nontoxic to recipients at the dosages and concentrations employed. The pharmaceutical formulations may contain materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, polyethylene glycol (PEG), sorbitan esters, polysorbates such as polysorbate 20 and polysorbate 80, Triton, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, REMINGTON'S PHARMACEUTICAL SCIENCES, 18.sup.th Edition, (A. R. Gennaro, ed.), 1990, Mack Publishing Company.

Optimal pharmaceutical formulations can be determined by one skilled in the art depending upon, for example, the intended route of administration, delivery format and desired dosage. See, for example, REMINGTON'S PHARMACEUTICAL SCIENCES, Id. Such formulations may influence the physical state, stability, rate of in vivo release and rate of in vivo clearance of the antibodies of the invention.

Primary vehicles or carriers in a pharmaceutical formulation can include, but are not limited to, water for injection, physiological saline solution or artificial cerebrospinal fluid, possibly supplemented with other materials common in compositions for parenteral administration. Neutral buffered saline or saline mixed with serum albumin are further exemplary vehicles. Pharmaceutical formulations can comprise Tris buffer of about pH 7.0-8.5, or acetate buffer of about pH 4.0-5.5, which may further include sorbitol or a suitable substitute. Pharmaceutical formulations of the invention may be prepared for storage by mixing the selected composition having the desired degree of purity with optional formulation agents (REMINGTON'S PHARMACEUTICAL SCIENCES, Id.) in the form of a lyophilized cake or an aqueous solution. Further, the formulations may be formulated as a lyophilizate using appropriate excipients such as sucrose.

Formulation components are present in concentrations that are acceptable to the site of administration. Buffers are advantageously used to maintain the composition at physiological pH or at a slightly lower pH, typically within a pH range of from about 5 to about 8.

The pharmaceutical formulations of the invention can be delivered parenterally. When parenteral administration is contemplated, the therapeutic formulations for use in this invention may be in the form of a pyrogen-free, parenterally acceptable aqueous solution. Preparation involves the formulation of the desired immunomicelle, which may provide controlled or sustained release of the product which may then be delivered via a depot injection. Formulation with hyaluronic acid has the effect of promoting sustained duration in the circulation.

Formulations may be formulated for inhalation. In these embodiments, a stealth immunomicelle formulation is formulated as a dry powder for inhalation, or inhalation solutions may also be formulated with a propellant for aerosol delivery, such as by nebulization. Pulmonary administration is further described in PCT Application No. PCT/US94/001875, which describes pulmonary delivery of chemically modified proteins and is incorporated by reference.

Formulations of the invention can be delivered through the digestive tract, such as orally. The preparation of such pharmaceutically acceptable compositions is within the skill of the art. Formulations disclosed herein that are administered in this fashion may be formulated with or without those carriers customarily used in the compounding of solid dosage forms such as tablets and capsules. A capsule may be designed to release the active portion of the formulation at the point in the gastrointestinal tract when bioavailability is maximized and pre-systemic degradation is minimized Additional agents can be included to facilitate absorption. Diluents, flavorings, low melting point waxes, vegetable oils, lubricants, suspending agents, tablet disintegrating agents, and binders may also be employed.

A formulation may involve an effective quantity of a micropoarticle containing formulation as disclosed herein in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

The pharmaceutical composition to be used for in vivo administration typically is sterile. In certain embodiments, this may be accomplished by filtration through sterile filtration membranes. In certain embodiments, where the composition is lyophilized, sterilization using this method may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

Once the formulation of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

Administration routes for formulations of the invention include orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. The pharmaceutical formulations may be administered by bolus injection or continuously by infusion, or by implantation device. The pharmaceutical formulations also can be administered locally via implantation of a membrane, sponge or another appropriate material onto which the desired molecule has been absorbed or encapsulated. Where an implantation device is used, the device may be implanted into any suitable tissue or organ, and delivery of the desired molecule may be via diffusion, timed-release bolus, or continuous administration.

Preferred methods of treatment and pharmaceutical formulations include the following.

In one embodiment, the invention provides a method of treating a subject who suffers from a cancer (any cancer as otherwise disclosed herein), preferably a cancer selected from the group consisting of breast cancer, ovarian cancer, colorectal cancer, glioblastoma multiform (GBM), melanoma, lung cancer and a glioma, the method comprising co-administering to the subject a pharmaceutically-effective amount of:

(a) one or more elements or compounds selected from the group consisting of arsenic, an arsenite and ATO (preferably, ATO);
(b) optionally, one or more chemotherapy agents (preferably, at least one DNA-damaging agent);
(c) optionally, radiation therapy; and
(b) further optionally, one or more PARP inhibitors. Preferably, ATO is used in combination with at least one DNA-damaging agent and/or radiation therapy. In certain further embodiments, an additional PARP inhibitor is also used (i.e., in the presence of absence of one or more chemotherapy agents and/or radiation therapy).

In a preferred embodiment, the subject is treated concomitantly by radiotherapy and the one or more elements or compounds selected from the group consisting of arsenic, an arsenite and ATO are administered to the subject as a radiosensitizer prior to or during radiotherapy.

In certain embodiments, the subject suffers from a treatment-resistant cancer selected from the group consisting of breast cancer in which BRCA1-deficient cells exhibit decreased sensitivity to PARP inhibitors; ovarian cancer which is resistant to platinum-containing anti-neoplastic drugs; hormone and castration-resistant prostate cancer; metastatic melanoma; drug resistant childhood acute lymphoblastic leukemia (ALL); and chemotherapy and radiotherapy-resistant glioblastomas, cervical cancer, esophageal cancer (EC), breast cancers and non-small cell lung cancer.

Preferably, the additional PARP inhibitor (i.e., other than an arsenic based PARP-1 inhibitor such as ATO as disclosed) is selected from the group consisting of NU1025; 3-aminobenzamide; 4-amino-1,8-naphthalimide; 1,5-isoquinolinediol; 6(5H)-phenanthriddinone; 1,3,4,5,-tetrahydrobenzo(c)(1,6)- and (c)(1,7)-naphthyridin-6 ones; adenosine substituted 2,3-dihydro-1H-isoindol-1-ones; AG14361; AG014699; 2-(4-chlorophenyl)-5-quinoxalinecarboxamide; 5-chloro-2-[3-(4-phenyl-3,6-dihydro-1 (2H)-pyridinyl)propyl]-4(3H)-quinazolinone; isoindolinone derivative INO-1001; 4-hydroxyquinazoline; 2-[3-[4-(4-chlorophenyl) 1-piperazinyl]propyl]-4-3(4)-quinazolinone; 1,5-dihydroxyisoquinoline (DHIQ); 3,4-dihydro-5 [4-(1-piperidinyl)(butoxy)-1(2H)-isoquinolone; CEP-6800; GB-15427; PJ34; DPQ; BS-201; AZD2281 (Olaparib); BS401; CHP101; CHP102; INH2BP; BSI201; BSI401; TIQ-A; an imidazobenzodiazepine; 8-hydroxy-2-methylquinazolinone (NU1025), CEP 9722, MK 4827, LT-673; 3-aminobenzamide; Olaparib (AZD2281; ABT-888 (Veliparib); BSI-201 (Iniparib); Rucaparib (AG-014699); INO-1001; A-966492; PJ-34; and the PARP1 inhibitors described in U.S. patent application Ser. No. 12/576,410.

One preferred embodiment provides a method of treating a subject who suffers from BRCA-associated breast or ovarian cancer, the method comprising co-administering to the subject a pharmaceutically-effective amount of:

(a) one or more elements or compounds selected from the group consisting of arsenic, an arsenite and ATO;
(b) at least one chemotherapy agent (preferably a DNA damaging agent), and
(c) Rucaparib and optionally an additional chemotherapy agent and/or one or more additional PARP inhibitors. The method may also be combined with radiation therapy to effect an intended therapeutic result.

Another preferred embodiment provides a method of treating a subject who suffers from one or more cancers selected from the group consisting of breast cancer, ovarian cancer and colorectal cancer, the method comprising co-administering to the subject a pharmaceutically-effective amount of:

(a) one or more elements or compounds selected from the group consisting of arsenic, an arsenite and ATO; and
(b) Olaparib and optionally an additional chemotherapy agent (preferably a DNA damaging agent) and/or one or more additional PARP inhibitors. This method may also be combined with radiation therapy to effect an intended therapeutic result.

Another preferred embodiment provides a method of treating a subject who suffers from one or more cancers selected from the group consisting of breast cancer, colorectal cancer, glioblastoma multiform (GBM) and melanoma, the method comprising co-administering to the subject a pharmaceutically-effective amount of:

(a) one or more elements or compounds selected from the group consisting of arsenic, an arsenite and arsenic trioxide (ATO); and
(b) Veliparib and optionally one or more chemotherapy agents (preferably a DNA damaging agent) and/or one or more additional PARP inhibitors.

Still another preferred embodiment provides a method of treating a subject who suffers from one or more cancers selected from the group consisting of breast cancer, colorectal cancer, glioblastoma multiform (GBM) and melanoma, the method comprising co-administering to the subject a pharmaceutically-effective amount of:

(a) one or more elements or compounds selected from the group consisting of arsenic, an arsenite and ATO; and
(b) Veliparib and optionally one or more chemotherapy agents (preferably, a DNA damaging agent and/or one or more additional PARP inhibitors. This method may also be combined with radiation therapy.

Another preferred embodiment provides a method of treating a subject who suffers from one or more cancers selected from the group consisting of breast cancer, lung cancer, a glioma and ovarian cancer, the method comprising co-administering to the subject a pharmaceutically-effective amount of:

(a) one or more elements or compounds selected from the group consisting of arsenic, an arsenite and ATO (preferably ATO); and
(b) gemcitabine and/or BSI-201 (Iniparib) and, optionally, one or more additional PARP inhibitors. This method may optionally be used in combination with radiation therapy.

Another preferred embodiment provides a method of treating a subject who suffers from a solid tumor, the method comprising co-administering to the subject a pharmaceutically-effective amount of:

(a) one or more elements or compounds selected from the group consisting of arsenic, an arsenite and ATO; and
(b) MK-4827 and optionally at least one chemotherapy agent (preferably, a DNA damaging agent) and/or one or more additional PARP inhibitors. This method also may be combined with radiation therapy.

The subject treated in the embodiment of the preceding paragraph may suffer from one or more cancers selected from the group consisting of breast cancer, ovarian cancer, non-small-cell lung cancer and prostate cancer.

In certain embodiments, a subject treated by the methods of treatment of the invention suffers from one or more cancers selected from the group consisting of relapsed or refractory T-cell prolymphocytic leukemia (T-PLL), chronic lymphocytic leukemia (CLL), locally advanced or metastatic colorectal carcinoma (CRC), persistent or recurrent endometrial carcinoma, locally advanced or metastatic triple negative or highly proliferative estrogen receptor positive (ER+) breast cancer and partially platinum-sensitive epithelial ovarian cancer.

Another preferred embodiment provides a method of treating a subject who suffers from a solid tumor, the method comprising co-administering to the subject a pharmaceutically-effective amount of:

(a) one or more elements or compounds selected from the group consisting of arsenic, an arsenite and ATO; and
(b) carboplatin and, optionally, MK-4827 and/or one or more additional PARP inhibitors. This method may also be used in combination with radiation therapy.

Still another preferred embodiment provides a method of treating a subject who suffers from an advanced solid tumor, the method comprising co-administering to the subject a pharmaceutically-effective amount of:

(a) one or more elements or compounds selected from the group consisting of arsenic, an arsenite and ATO; and
(b) CEP-9722 and optionally, at least one chemotherapy agent (preferably, a DNA damaging agent) and/or one or more additional PARP inhibitors. This method may also be used in combination with radiation therapy.

Another preferred embodiment provides a method of treating a subject who suffers from melanoma, the method comprising co-administering to the subject a pharmaceutically-effective amount of:

(a) one or more elements or compounds selected from the group consisting of arsenic, an arsenite and ATO; and
(b) temozolomide in combination with E7016 and/or INO-1001 and, optionally at least one chemotherapy agent (one or more additional PARP inhibitors. This method may also be used with radiation therapy.

Another preferred embodiment provides a method of treating a subject who suffers from metastatic germline BRCA mutated breast cancer, the method comprising co-administering to the subject a pharmaceutically-effective amount of:

(a) one or more elements or compounds selected from the group consisting of arsenic, an arsenite and ATO; and
(b) BMN-673 and, optionally, at least one chemotherapy agent (preferably, a DNA damaging agent) and/or one or more additional PARP inhibitors. This method may also be used in combination with radiation therapy.

Still another preferred embodiment provides a method of treating a subject who suffers from metastatic breast cancer and/or ovarian cancer, the method comprising co-administering to the subject a pharmaceutically-effective amount of:

(a) one or more elements or compounds selected from the group consisting of arsenic, an arsenite and ATO; and
(b) Rucaparib and optionally, at least one chemotherapy agent (preferably, a DNA damaging agent) and/or one or more additional PARP inhibitors. This method may also be used in combination with radiation therapy. This method may also be used in combination with radiation therapy.

Another preferred embodiment provides a method of treating a subject who suffers from metastatic melanoma and/or breast cancer, the method comprising co-administering to the subject a pharmaceutically-effective amount of:

(a) one or more elements or compounds selected from the group consisting of arsenic, an arsenite and ATO; and
(b) CEP 9722 and optionally, at least one chemotherapy agent (preferably, a DNA damaging agent) and/or one or more additional PARP inhibitors. This method may also be used in combination with radiation therapy.

Another preferred embodiment provides a method of treatment comprising treating a subject who suffers from non-small-cell lung cancer (NSCLC), the method comprising co-administering to the subject a pharmaceutically-effective amount of:

(a) one or more elements or compounds selected from the group consisting of arsenic, an arsenite and ATO; and
(b) Veliparib (ABT-888) and, optionally at least one chemotherapy agent (preferably, a DNA damaging agent) and/or one or more additional PARP inhibitors. This method may also be used in combination with radiation therapy.

Another preferred embodiment provides a method of treating a subject who suffers from one or more cancers selected from the group consisting of breast cancer, colorectal cancer, glioblastoma multiform (GBM) and melanoma, the method comprising co-administering to the subject a pharmaceutically-effective amount of:

(a) one or more elements or compounds selected from the group consisting of arsenic, an arsenite and arsenic trioxide (ATO); and
(b) 3-aminobenzamide and, optionally at least one chemotherapy agent (preferably, a DNA damaging agent) and/or one or more additional PARP inhibitors. This method may also be used in combination with radiation therapy.

In the methods of treatment described herein, the subject may also be treated with one or more platinum-based antineoplastic drugs, e.g. an antineoplastic drug selected from the group consisting of Cisplatin, Carboplatin, Oxaliplatin, Satraplatin, Picoplatin, Nedaplatin, Triplatin tetranitrate, and Lipoplatin.

One example of a pharmaceutical formulation of the invention comprises a pharmaceutically-effective amount of:

(a) one or more elements or compounds selected from the group consisting of arsenic, an arsenite and ATO;
(b) one or more PARP inhibitors (preferably one or more PARP-1 inhibitors);
(c) optionally, one or more platinum-based anti-neoplastics; and
(d) optionally, a pharmaceutically-acceptable excipient.

These and other aspects of the invention are illustrated further in the following non-limiting Example.

### Example 1

### Differential Binding of Monomethylarsonous Acid Compared to Arsenite and Arsenic Trioxide with Zinc Finger Peptides and Proteins

### Experimental Procedures

### Chemicals

Peptides derived from the finger motifs of APTX, XPA and the first zinc finger motif of PARP-1 (sequences in Table 1) were commercially synthesized by Genemed Synthesis Inc. (San Antonio, Tex.). Purity confirmed by HPLC was greater than 95%. Diiodomethylarsine (MMAIII iodide, CH₃AsI₂, >98% pure) was prepared by the Synthetic Chemistry Facility Core (Southwest Environmental Health Sciences Center, Tucson, Ariz.) and kindly provided by Dr. A. Jay Gandolfi, University of Arizona. As₂O₃ (ATO, >99.95%) was obtained from Mallinckrodt Chemical Works (St. Louis, Mo.). Cobalt chloride, zinc chloride, and sodium arsenite were obtained from Fluka Chemie. All other chemicals were obtained from Sigma-Aldrich.

### Cobalt Spectrometry Analysis of Free Metal Binding Sites on Zinc Finger Peptides.

Lyophilized zinc finger peptides were suspended at 1 mM in 20 mM Tris (pH 7.8) containing 0.1 mM Tris(2-carboxyethyl)phosphine (TCEP), to protect the cysteine residues from oxidation prior to incubations. Solutions of arsenic compounds were prepared freshly in 20 mM Tris (pH 7.8) before incubation with zinc finger peptides. Zinc finger peptides diluted to 100 μM were incubated with 50, 100 or 200 μM arsenic compounds at room temperature for 30 min, then cobalt chloride was added to a final concentration of 200 μM. The absorption spectra from 260 to 800 nm were collected at 25° C. on a SpectraMax M2 spectrophotometer (Molecular Devices, LLC, Sunnyvale, Calif.). Absorbance at 660 nm indicates the formation of a cobalt and zinc finger peptide complex,^{1,22,23} and therefore, the cobalt spectrum A₆₆₀ value represents the amount of sites on zinc fingers that are still available for metal ions to bind after treatments of arsenic compounds.

### UV-Vis Spectrometry Analysis of As—S Bond Formation on Zinc Finger Peptides.

Aliquots of 100 μM zinc finger peptides in 20 mM Tris (pH 7.8) were incubated with various concentrations (0-200 μM) of arsenic compounds for 30 min at 25° C., then the UV-Vis absorption spectra of the mixtures from 260 to 500 nm were collected at 25° C. on a SpectraMax M2 spectrophotometer (Molecular Devices, LLC, Sunnyvale, Calif.). A₂₇₀ is used as the indication of an As—S bond formation due to arsenic interaction with cysteine residues on zinc finger peptides.^{8-11,24}

### Mass Spectrometry Analysis

Lyophilized peptides were suspended at a concentration of 1 mM in 20 mM Tris (pH 7.8) containing 0.1 mM TCEP to protect the cysteine residues from oxidation. Stock solutions of arsenic compounds were freshly prepared at a concentration of 1 M in 20 mM Tris (pH 7.8). Aliquots of 100 μM zinc finger peptides were incubated with 100 μM arsenic compounds for 30 min at 25° C. The samples were then diluted 50 times in 5 mg/mL α-cyano-4-hydroxycinnamic acid (Sigma-Aldrich) in a 1:1 (v/v) water/acetonitrile solution, and 1 μL of each sample was deposited in duplicate on the MALDI plate, allowed to dry at 37° C., and MALDI-TOF-MS analyses performed on an Applied Biosystems 4700 Proteomics Analyzer (TOF/TOF) operating in MS reflector-positive ion mode. The total acceleration voltage was 20 kV. Desorption was performed using a neodymium/yttrium-aluminum-garnet laser (355 nm, 3 ns pulse width, and 200 Hz repetition rate). Mass spectra were acquired with laser pulses over a mass range of m/z from 1000 to 5000 Da using focus mass of 3500. Final mass spectra were the summation of 10 subspectra, each acquired with 200 laser pulses.

### Intrinsic Fluorescent Analysis of Arsenical Binding to Zinc Fingers

Aliquots of 100 μM zinc finger peptides were incubated with different concentrations of arsenic compounds or 100 μM zinc chloride for 30 min at 25° C. After that, the emission fluorescent spectra from 300 to 400 nm were collected at 25° C. on a SpectraMax M2 fluorescent spectrophotometer (Molecular Devices, LLC, Sunnyvale, Calif.). The excitation wavelength was 280 nm. The intensity of fluorescence is related to the chemical environments of phenylalanine, tyrosine, and tryptophan. The intrinsic fluorescence intensity of zinc finger peptides undergoes a dramatic change on folding/unfolding. This allows tertiary structure change of zinc finger peptides to be monitored by fluorescence spectroscopy. Fluorescent intensity at 350 nm was used to represent the status of the tertiary structure of zinc finger peptides with different treatments.

### Cell Culture and Zinc Finger Protein Isolation by Immunoprecipitation.

The human keratinocyte cell line (HaCaT) was a kind gift from Dr. Mitch Denning (Loyola University Medical Center, Maywood, Ill.). Cells were maintained as described previously.^{12-14,16} After exposure to 2 μM arsenic compounds for 24 h, cells were harvested in RIPA cell lysis buffer (25 mM Tris-HCl pH 7.6, 150 mM NaCl, 1% NP-40, 1% sodium deoxycholate, 0.1% SDS), sonicated, and centrifuged at 14,000 rpm for 15 min at 4° C. to remove cellular debris. Protein (500 μg in 500 μl) was incubated with 5 μL of rabbit polyclonal antibody (APTX, Abcam #31841; PARP-1, Cell Signaling #9542 or XPA, Abcam ab85914) for at least 2 h at 4° C. Protein A beads (Invitrogen) were added in a 1:1 slurry, and samples were incubated for an additional 2 h at 4° C. The beads were recovered by centrifugation at 10,000 rpm for 5 min at 4° C. and washed five times with 1 mL of lysis buffer. To elute protein, the pellets were incubated with 100 μL of 100 mM citric acid (pH 3.0) for 30 min, followed by centrifugation at 14,000 rpm for 5 min at 4° C. The supernatant was adjusted to pH=7 with 10 M NaOH.

### Measurement of Zinc Content in Protein

Proteins obtained from cells by immunoprecipitation were incubated with 10 mM H₂O₂ for at least 2 h at 4° C. to release zinc from proteins. Zinc content was measured by adding 10 μL of 1 mM 4-(2-pyridylazo)resorcinol to 100 μL of protein sample followed by scanning the UV-Vis spectra at 350 to 550 nm on a SpectraMax M2 spectrophotometer (Molecular Devices, LLC, Sunnyvale, Calif.). The absorbance of resorcinol shifts from 411 to 493 nm in the presence of zinc, and the 493 nm peak is recorded and compared with a standard curve for calculation of zinc content in protein samples.^{21,23}

### Results

### Cobalt Spectrometry Analysis of Differential Selectivity of Occupying Metal Binding Sites on Zinc Fingers

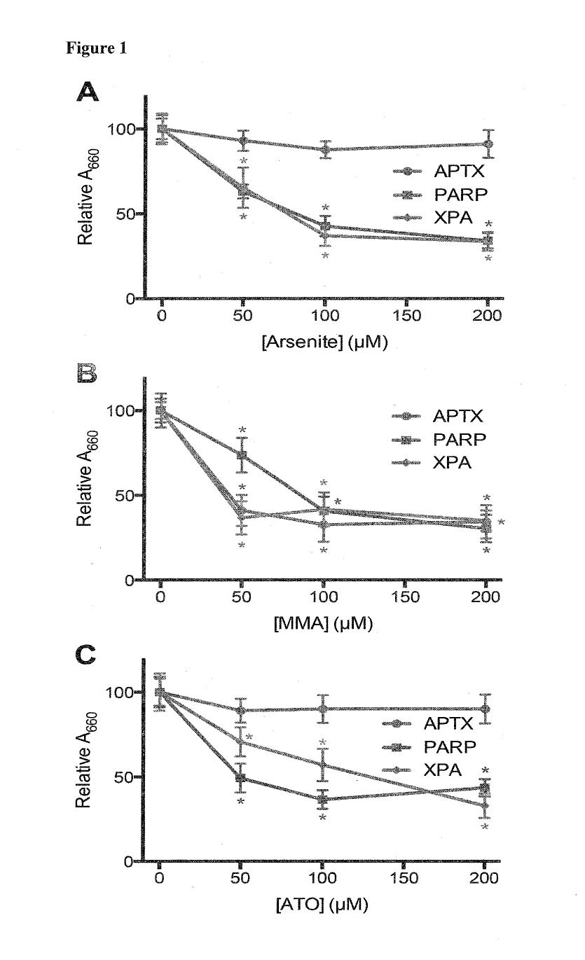

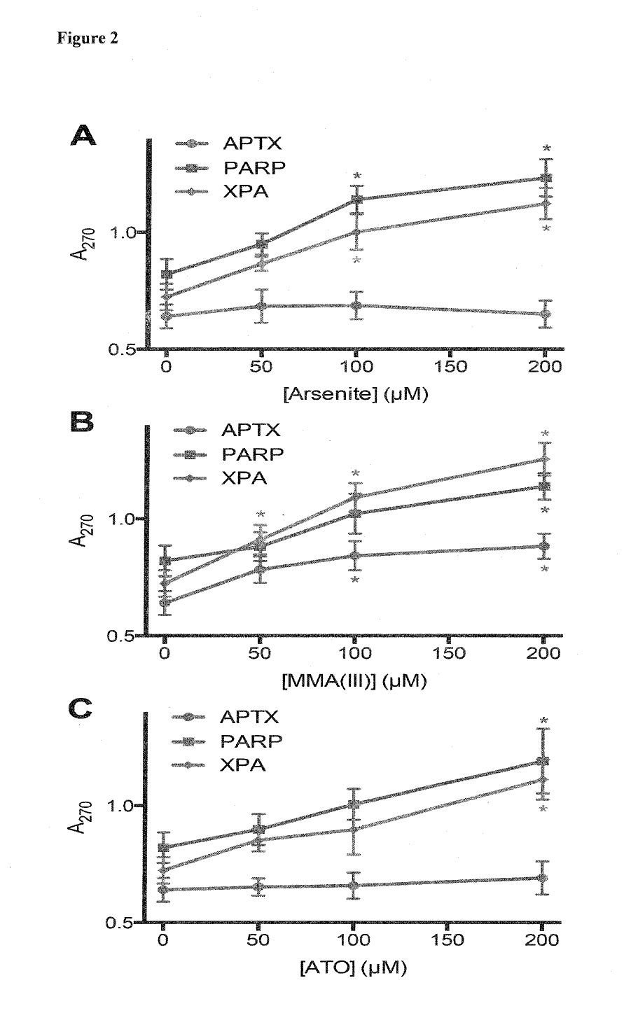

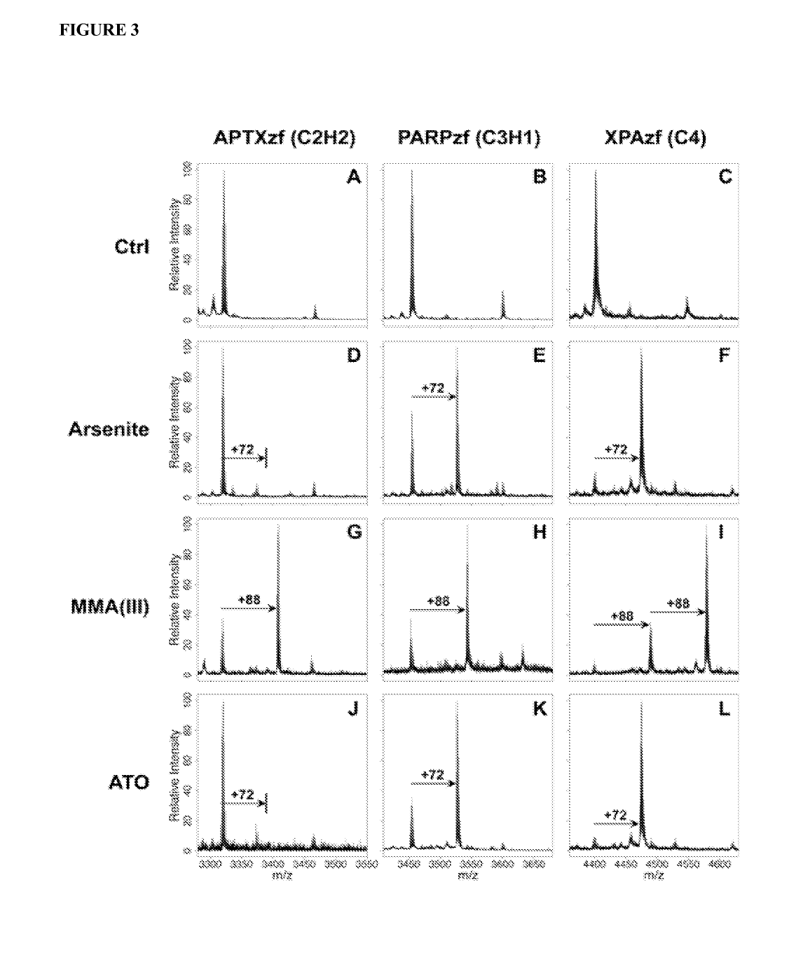

Zinc binding within zinc finger motifs is critical for the maintenance of the tertiary structure and activity of zinc finger proteins. Occupation of metal binding sites by arsenic is an indicator of arsenic interaction with zinc fingers. To determine whether an arsenic compound is capable of occupying the metal binding sites and to investigate differences in metal binding site occupation by MMA(III), arsenite or ATO, we used cobalt as a probe to detect available metal binding sites on zinc finger motifs after incubation with arsenic compounds. Cobalt binding with zinc finger motifs generates absorbance at 660 nm,^{22,23} which is used to quantitatively determine the remaining free metal binding sites after arsenic occupation. The zinc finger peptides derived from the zinc finger domains of C2H2 (APTX), C3H1 (PARP-1), and C4 (XPA) were incubated with arsenicals, and then the cobalt spectrum was analyzed. In PARP-1 and XPA peptides, arsenite incubation decreased subsequent cobalt binding in a concentration-dependent manner, but not in APTX peptides (FIG. 1A). This indicates that arsenite selectively occupies the metal binding site on C3H1 and C4, but not the C2H2 zinc fingers. This result is consistent with our published findings.²¹ In contrast, incubation of peptide with MMA(III) led to a concentration-dependent decrease in cobalt binding to all three peptides indicating that MMA(III) occupied the metal binding sites on all three configurations of zinc fingers (FIG. 1B). ATO showed the same selectivity as arsenite (FIG. 1C). These results demonstrate that arsenite and ATO selectively occupy metal binding sites on C3H1 and C4 zinc fingers, while MMA(III) occupies metal binding sites on each of the C2H2, C3H1, and C4 zinc finger peptides.
UV-Vis Spectral Analysis of Differential Selectivity of As—S Bond Formation with Zinc Fingers
When arsenicals occupy metal binding sites on zinc fingers, they coordinate with cysteine residues, forming an As—S bond. Therefore, the As—S bond is a key structure of arsenic interacting with the thiol group on cysteine residues of zinc fingers. In order to determine whether arsenic interaction with thiol groups is consistent with binding site occupation on zinc finger motif, we analyzed As—S bond formation between arsenic and cysteine residues on zinc finger peptide. In the UV-Vis spectrum, an As—S bond can generate absorbance from 260 to 340 nm.²⁴ We used A₂₇₀ in UV-Vis spectra as the indicator of As—S bond formation. Varying concentrations of arsenic compounds were incubated with 100 μM of different configurations of zinc finger peptides for 30 min at room temperature, and the UV-Vis spectra of the mixtures were recorded. In arsenite treated samples, A₂₇₀ values increased in a concentration-dependent manner for PARP-1 and XPA zinc finger peptides, but not for APTX (FIG. 2A), showing that arsenite selectively forms As—S bond with C3H1 and C4 zinc fingers, as expected from our previous report.²¹ In contrast, in MMA(III) treated samples, A₂₇₀ values increased for all three types of zinc fingers in a MMA(III) concentration dependent manner (FIG. 2B), indicating that MMA(III) could form As—S bond with each zinc finger. ATO formed As—S bonds with PARP-1 and XPA zinc fingers, but not APTX (FIG. 2C), showing the same binding selectivity for C3H1 and C4 configurations as arsenite in terms of forming As—S bonds. These results indicate that interaction with Cys residues by forming As—S bonds is the molecular mechanism for zinc binding site occupation by arsenic. Among the three arsenicals, arsenite and ATO showed the same selectivity in forming As—S bonds with C3H1 and C4 zinc fingers, but MMA(III) could form As—S bonds with all three configurations of zinc finger peptides.
Mass Spectrometry Analysis of Arsenicals Interacting with C2, C3, and C4 Zinc Fingers
To further understand the differences in binding selectivity, MALDI-TOF mass spectrometry was utilized to analyze the precise molecular weights of the arsenic-zinc finger complex. Zinc finger peptides with different configurations (100 μM) were treated with 100 μM arsenic compounds. The mass spectra of apo-zinc finger peptide for APTX, PARP-1, and XPA are shown in FIGS. 3A, B, and C, respectively. Arsenite showed no binding to the APTX zinc finger (FIG. 3D), but bound with PARP-1 and XPA zinc finger peptides, both giving +72 m/z shift against the apo-peptide signals (FIGS. 3E & F), indicating that arsenite coordinates with zinc fingers with the arsenic atom alone (m/z=75), releasing three hydrogen atoms (m/z=−3) at the same time. This result is consistent with our previous published data.²¹ MMA(III) induced a +88 m/z shift to the APTX zinc finger peptide (FIG. 3G). The interpretation of a +88 m/z shift is that MMA(III) bound to APTX zinc finger peptide with As—CH₃ (m/z: 75, As+12, C+3, 3H=90), losing 2H (m/z: −2) from Cys residues on zinc finger peptides. MMA(III) also bound to the PARP-1 zinc finger (FIG. 3H) with a +88 m/z shift, showing that MMA(III) used 2 cysteine residues for binding. For XPA, 1 molecule of MMA(III) bound to the XPA zinc finger, giving a +88 m/z shift to the apo-peptide. At the same time, we detected the signal of 2 molecules of MMA(III) bound to the same XPA zinc finger peptide (FIG. 3I), giving a +88 m/z shift for each. Since the XPA zinc finger has 4 cysteine residues, when 1 molecule of MMA(III) bound to the zinc finger peptide, occupying 2 cysteine residues, there were still 2 free cysteine residues available for another molecule of MMA(III) coordination. This result further confirms that, unlike arsenite, MMA(III) only occupies 2 Cys during binding with zinc fingers. The mass spectra for ATO was the same as arsenite; it did not bind to the APTX zinc finger (FIG. 3J), but bound with PARP-1 or XPA zinc fingers, giving a +72 m/z shift (FIGS. 3K & L). Therefore, the selectivity of ATO binding with zinc fingers was the same as arsenite. Furthermore, the +72 m/z shift indicates that ATO bound with zinc fingers in the same manner as arsenite, i. e. coordinating with 3 Cys residues. Together, the mass spectrometry results show that MMA(III) coordinated with 2 Cys, but arsenite and ATO both occupy 3 Cys when binding with zinc fingers.

### Intrinsic Fluorescence Analysis of Alternation of Tertiary Structure of Zinc Fingers

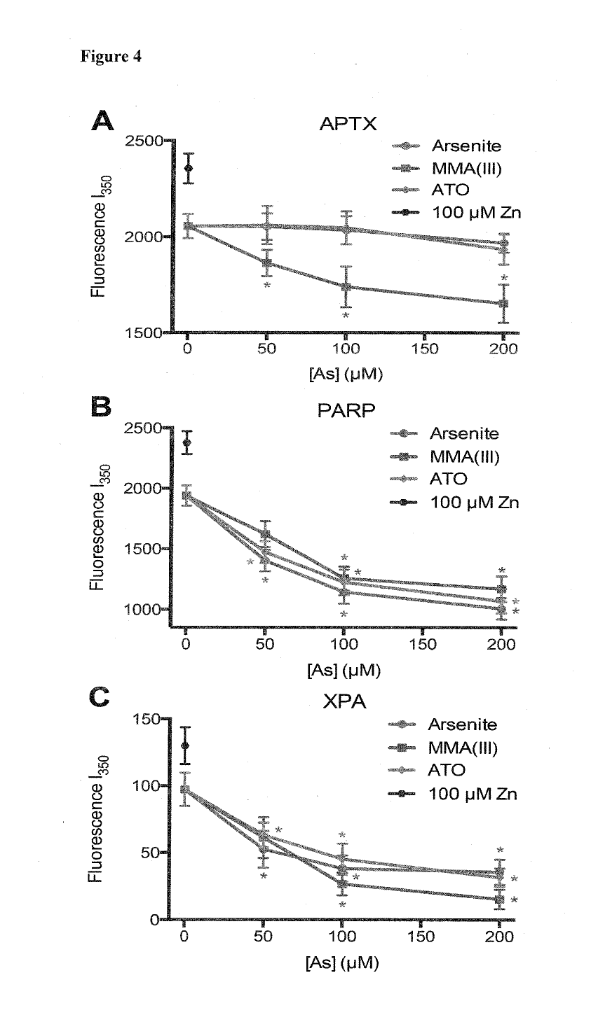

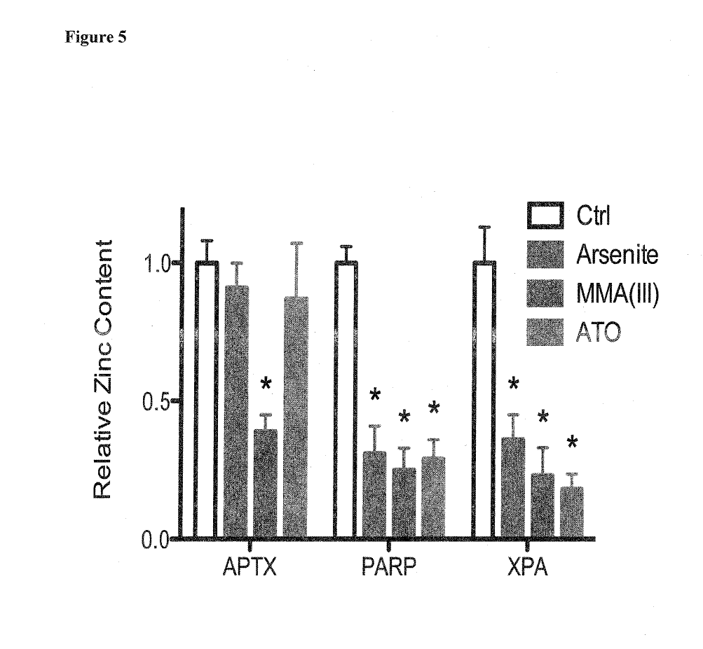

Next we investigated whether arsenic binding could lead to structural changes of the zinc finger peptides. Zinc finger motifs of DNA repair proteins are frequently responsible for DNA recognition and DNA binding.^{25,26} Maintaining a correct tertiary structure is critically important for DNA binding and DNA repair capability. In order to investigate conformational changes due to arsenic binding to the zinc fingers, intrinsic fluorescence was used to analyze tertiary structure alteration on zinc finger peptides after treatments with arsenic compounds, as compared to zinc incubation. Intrinsic fluorescence is primarily generated from tryptophan and tyrosine residues (phenylalanine also contributes a small portion), representing the chemical environment of these amino acids. The intensity of fluorescence usually increases while peptides fold and side chains of Trp and Tyr are located in a relatively hydrophobic environment.²⁷ We treated different configurations of zinc finger peptides (100 μM) for 30 min at room temperature with varying concentrations of arsenic compounds. After that, we collected the fluorescent spectra of each sample under the excitation wavelength of 280 nm, emission from 300 to 400 nm. Treatment with 100 μM zinc chloride was used as a control to show the natural folded conformation of the zinc finger peptides. Finally, fluorescent intensity at 350 nm was used to represent the tertiary structure change of zinc fingers. As shown in FIG. 4A, fluorescent signal of APTX zinc finger could be decreased in a concentration-dependent manner only by MMA(III), but not arsenic or ATO, while zinc treatment generated the highest fluorescent signal (shown as a single data point in top left corner of FIGS. 1A, 1B, and 1C). This result indicates APTX zinc finger forms a defined structure with zinc ions, but MMA(III) treatment could unfold the structure in a concentration dependent manner. Arsenite or ATO showed no effect, which is consistent with the lack of binding based on the selectivity data (FIGS. 1, 2, and 3). For the PARP-1 zinc finger peptide, all 3 arsenic compounds decreased the fluorescent intensity in a concentration dependent manner (FIG. 4B). Results on XPA zinc finger exhibited a similar trend (FIG. 4C) as PARP-1 (FIG. 4B). Together, these results indicate that MMA(III) alters the tertiary structure of all 3 conformations of zinc fingers, while arsenic and ATO selectively disrupted the tertiary structure of C3H1 and C4 zinc fingers. Furthermore, the findings demonstrate that the alteration in the tertiary structure of zinc finger is a direct consequence of arsenic binding, and that the selectivity of structural changes induced by arsenicals is consistent with the binding selectivity.
Selective Loss of Zinc from Zinc Finger Proteins in Cells Exposed to Arsenicals
Finally, in order to test whether the selectivity and behavior of MMA(III), arsenite and ATO binding with zinc finger proteins are applicable in cells, zinc content in DNA repair proteins from cells treated with arsenicals was analyzed. We have reported that zinc loss from zinc finger proteins is a direct consequence of arsenic binding and a key event for protein function loss and arsenic toxicity in cells.²⁰ Human keratinocyte (HaCat) cells were treated with 2 μM of arsenite, ATO or MMA(III) for 24 h. APTX, PARP-1, and XPA protein were immunoprecipitated from cell extracts using corresponding antibodies, and the zinc content in each protein sample was determined. As shown in FIG. 5, MMA(III) caused zinc loss from all three configurations of zinc finger proteins, while arsenite and ATO selectively displaced zinc from PARP-1 and XPA proteins isolated from cells. These results demonstrate that arsenite and ATO selectively interacted with C3H1 and C4 zinc fingers in the context of native protein, but MMA(III) interacted with all 3 configurations of zinc finger proteins in HaCat cells.

### DISCUSSION

Targeted interaction with zinc finger domains is considered an important mechanism for arsenic toxicity and co-carcinogenesis. In the present study, by using cobalt spectrometry, we demonstrated that both inorganic and organic arsenicals interacted with zinc fingers by direct occupation of metal binding sites. UV-vis spectra demonstrated that all three arsenicals formed As—S bonds with Cys residues on zinc fingers, illustrating the importance of Cys residues for arsenic binding. Mass spectrometry analysis further confirmed the formation of As—S covalent bond. In addition, loss of hydrogen atoms on the complexes confirmed that arsenicals interacted with Cys residues but not His residues on zinc fingers, which is different from the mechanism of zinc binding with zinc fingers. This may also be one of the reasons that arsenic binding changes conformation of zinc fingers, which we demonstrated by intrinsic fluorescent analysis. Collectively, from the data offered by these diverse techniques, we show that arsenite, MMA(III), and ATO occupy metal binding sites on zinc fingers by directly coordinating with Cys residues to form As—S bonds, leading to conformational change as well as zinc loss from the zinc fingers.

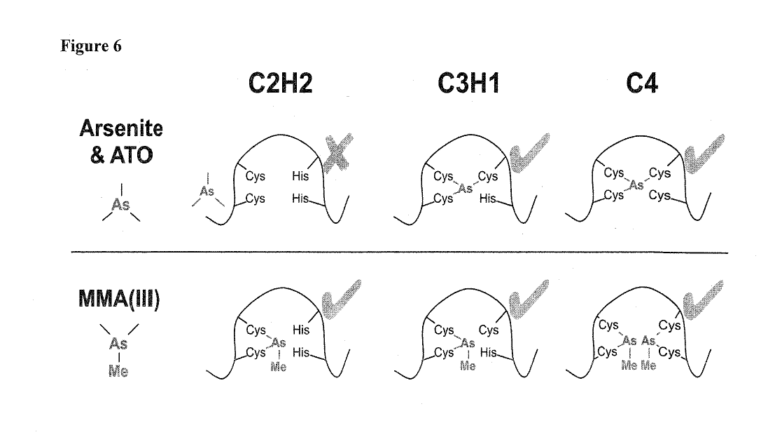

For the two trivalent inorganic arsenicals, arsenite and ATO, their patterns of interaction with zinc fingers are the same. This conclusion is drawn from the cobalt spectra showing the occupation of metal binding sites, the UV-Vis spectra showing the formation of As—S bonds, and the mass spectra showing the coordination with 3 Cys residues on zinc fingers. As shown by mass spectrometry, arsenite and ATO gave exactly the same +72 m/z shift to C3H1 or C4 zinc fingers, indicating that both arsenite and ATO bind to zinc fingers using the arsenic atom only, and coordinate with 3 Cys on zinc finger motif, with the release of three hydrogens. This behavior may explain the selectivity in binding with C3H1 and C4 zinc fingers (as illustrated in FIG. 6, top row). Kitchin and Wallace reported that arsenite bound C3H1 and C4 complexes are over 2 orders of magnitude more stable than a C2H2 complex (155 versus 1.29 min) in kinetic studies.²⁸ Therefore, it is reasonable to suggest that trivalent arsenite or ATO may form two As—S bonds with C2H2 zinc fingers while leaving the third bond unoccupied, but the resulting product is too unstable to accumulate to high enough concentration to be detected by our analytical approaches due to the presence of an unoccupied bond. This binding selectivity toward zinc fingers with 3 or more Cys could have potential biological significance. In terms of structural/functional consequences, arsenite and ATO led to selective conformational changes of C3H1 and C4 zinc fingers and induced zinc loss selectively in PARP-1 and XPA proteins in cells. There was no significant difference in the efficiency of arsenite and ATO in changing the structure of zinc fingers as well as zinc release from proteins. Circular dichroism (CD) and nuclear magnetic resonance (NMR) spectra indicate that arsenic binding to a C3H1 or C4 zinc finger motif result in an unfolded structure.^{29,30} These results, together with the findings here, provide evidence to the alteration of zinc finger structure by arsenic binding and further support that arsenic interaction with zinc finger proteins will likely disrupt protein function. Since C3H1 and C4 zinc finger proteins are a minority in the whole zinc finger protein family (less than 20%), inorganic arsenicals could target some C3H1 and C4 zinc finger proteins in cells more effectively even at low concentrations.

It has been reported that that MMA(III) binds to PARP-1 and XPA zinc finger peptides.^{31,32} Here we further demonstrated that MMA(III) could occupy metal binding sites (FIG. 1A) and form As—S bonds (FIG. 2A) on all 3 configurations of zinc fingers, non-selectively. This non-selective interaction is also confirmed structurally and functionally using conformation and zinc content analysis (FIGS. 4 and 5). The molecular mechanism behind the non-selectivity is that MMA(III) covalently binds to two Cys residues, releasing two hydrogen, as shown in mass spectrometry (FIGS. 3G, H, and I). In contrast to arsenite and ATO binding with three Cys, MMA(III) only binds with two Cys on zinc fingers. This is demonstrated by the +88 m/z shift, as well as binding of two MMA(III) molecules to C4 zinc finger (FIG. 3I). The +88 m/z shift and loss of 2H is consistent with previous findings by Wnek et. al.³¹ And a complex of two MMA(III) molecules with the C4 XPA zinc finger peptide has been detected by Piatek et. al.³² In this work, we confirmed these findings using MALDI-MS and put these together to explain the mechanism of differential binding of MMA(III) to zinc finger peptides at the molecular level. Although arsenic in MMA(III) is still trivalent, one bond is already occupied by the methyl group, leaving remaining two bonds for binding with Cys. The +88 m/z shift in mass spectrometry studies with C2H2, C3H1, and C4 zinc fingers confirm that when MMA(III) binds to zinc fingers, the methyl group is still bound to arsenic. In other words, presence of the methyl group on MMA(III) changed the binding behavior and selectivity of this trivalent arsenical (as illustrated in FIG. 6, bottom row), enabling it to bind with two Cys on zinc fingers. Importantly, unlike the situation with inorganic arsenite, the product derived from MMA(III) binding with two Cys is stable since there is no longer an un-occupied bond existing on the molecule. This result indicates that the methylation of arsenic could dramatically change the preference and profile of arsenic interaction with zinc finger proteins. And different binding selectivity may lead to different toxicity and carcinogenesis effect. Some recent studies showed that MMA(III) is more toxic than inorganic arsenic in terms of certain parameters of carcinogenesis, such as cell transformation.^{33,34} As for the possible molecular mechanism, Piatek et. al, demonstrated that MMA(III) acts more effectively than arsenite in destroying the structure of C4 zinc fingers.³² Meanwhile, in studies of PARP activity, up to 90% inhibition is readily evident at sub-μM concentration of arsenite in human keratinocytes.^{16,21} But exposure to 1 μM MMA(III) caused about 30% PARP activity inhibition in urothelial cells.³¹ These findings might suggest that arsenite causes a greater magnitude of PARP inhibition than MMA(III), or may simply due to different cell types. Apparently, further research is needed to investigate the relationship between binding selectivity and toxicity/carcinogenesis, i. e, whether the change of selectivity enhances the effect of arsenic in vivo or simply dilutes arsenic across the large family of zinc finger proteins.

In conclusion, this work demonstrates that arsenite and ATO have the same selective effect in binding with C3H1 and C4 zinc finger proteins, whereas MMA(III) interacts with all three configurations of zinc finger proteins. Methylation of trivalent inorganic arsenicals is responsible for the change in binding selectivity. These findings provide insightful understanding for the molecular mechanisms underlying the differential effects of inorganic versus methylated arsenicals, as well as the role of in vivo arsenic methylation in arsenic toxicity and carcinogenesis.

### Funding Sources

This work was supported by grants from the U.S. National Institutes of Health (R01ES15826 and R01ES021100). Support was also provided by the UNM Cancer Center P30CA118100 through a post-doc matching grant 1127 and a pilot award 1118. The MMA(III) was prepared in the Synthetic Core of the Southwest Environmental Health Sciences Center (P30ES006694) and supplied by the University of Arizona Superfund Program (ES 04940).

### Acknowledgements

We thank Dr. Karen L. Cooper for valuable discussion.

### ABBREVIATIONS

MMA(III), monomethylarsonous acid;
ATO, arsenic trioxide;
PARP-1, poly(ADP-ribose) polymerase-1;
APTX, aprataxin;
XPA, Xeroderma pigmentosum group A
UV-Vis spectroscopy, Ultraviolet-visible spectroscopy
MALDI-TOF-MS, Matrix-assisted laser desorption/ionization time-of-flight mass spectrometry

TABLE 1
Sequences of zinc finger peptides.

| Name | Sequence |
|---|---|
| APTX | PLRCHECQQLLPSIPQLKEHLRKHWTQ |
| (C2H2) | |
| PARP-1 | GRASCKKCSESIPKDKVPHWYHFSCFWKV |
| ZF 1 | |
| (C3H1) | |
| XPA (C4) | DYVICEECGKEFMDSYLMNHFDLPTCDNCRDADDKHK |

### REFERENCES

(1) Agency for Toxic Substances Disease Registry (ATSDR). (2007) Toxicological profile for Arsenic. U.S. Department of Health and Human Services, Public Health Service.
(2) Schuhmacher-Wolz, U., Dieter, H. H., Klein, D., and Schneider, K. (2009) Oral exposure to inorganic arsenic: evaluation of its carcinogenic and non-carcinogenic effects. Crit. Rev. Toxicol. 39, 271-298.
(3) Rahman, M. (2006) Prevalence of arsenic exposure and skin lesions. A population based survey in Matlab, Bangladesh. J Epidemiol. Community Health 60, 242-248.
(4) Schoen, A., Beck, B., Sharma, R., and Dubé, E. (2004) Arsenic toxicity at low doses: epidemiological and mode of action considerations. Toxicol. Appl. Pharmacol. 198, 253-267.
(5) Yoshida, T., Yamauchi, H., and Fan Sun, G. (2004) Chronic health effects in people exposed to arsenic via the drinking water: dose-response relationships in review. Toxicol. Appl. Pharmacol. 198, 243-252.
(6) World Health Organization. (2006) Guidelines for drinking-water quality. World Health Organization.
(7) Burchiel, S. W., Mitchell, L. A., Lauer, F. T., Sun, X., McDonald, J. D., Hudson, L. G., and Liu, K. J. (2009) Immunotoxicity and biodistribution analysis of arsenic trioxide in C57Bl/6 mice following a 2-week inhalation exposure. Toxicol. Appl. Pharmacol. 241, 253-259.
(8) Aposhian, H. V., Zakharyan, R. A., Avram, M. D., Sampayo-Reyes, A., and Wollenberg, M. L. (2004) A review of the enzymology of arsenic metabolism and a new potential role of hydrogen peroxide in the detoxication of the trivalent arsenic species. Toxicol. Appl. Pharmacol. 198, 327-335.
(9) Dopp, E., Kligerman, A. D., and Diaz-Bone, R. A. (2010) Organoarsenicals. Uptake, metabolism, and toxicity. Met. Ions Life Sci. 7, 231-265.
(10) Drobná, Z., Walton, F. S., Harmon, A. W., Thomas, D. J., and Stýblo, M. (2010) Interspecies differences in metabolism of arsenic by cultured primary hepatocytes. Toxicol. Appl. Pharmacol. 245, 47-56.
(11) Hirner, A. V., and Rettenmeier, A. W. (2010) Methylated metal(loid) species in humans. Met. Ions Life Sci. 7, 465-521.
(12) Styblo, M., Del Razo, L. M., Vega, L., Germolec, D. R., LeCluyse, E. L., Hamilton, G. A., Reed, W., Wang, C., Cullen, W. R., and Thomas, D. J. (2000) Comparative toxicity of trivalent and pentavalent inorganic and methylated arsenicals in rat and human cells. Arch. Toxicol. 74, 289-299.
(13) Kligerman, A. D., Doerr, C. L., Tennant, A. H., Harrington-Brock, K., Allen, J. W., Winkfield, E., Poorman-Allen, P., Kundu, B., Funasaka, K., Roop, B. C., Mass, M. J., and DeMarini, D. M. (2003) Methylated trivalent arsenicals as candidate ultimate genotoxic forms of arsenic: induction of chromosomal mutations but not gene mutations. Environ. Mol. Mutagen. 42, 192-205.
(14) Eblin, K. E., Bredfeldt, T. G., and Gandolfi, A. J. (2008) Immortalized human urothelial cells as a model of arsenic-induced bladder cancer. Toxicology 248, 67-76.
(15) Quintal, S. M., dePaula, Q. A., and Farrell, N. P. (2011) Zinc finger proteins as templates for metal ion exchange and ligand reactivity. Chemical and biological consequences. Metallomics 3, 121-139.
(16) Ding, W., Liu, W., Cooper, K. L., Qin, X. J., de Souza Bergo, P. L., Hudson, L. G., and Liu, K. J. (2008) Inhibition of poly(ADP-ribose) polymerase-1 by Arsenite interferes with repair of oxidative DNA damage. J Biol. Chem. 284, 6809-6817.
(17) Asmuss, M. (2000) Differential effects of toxic metal compounds on the activities of Fpg and XPA, two zinc finger proteins involved in DNA repair. Carcinogenesis 21, 2097-2104.
(18) Hartwig, A., Blessing, H., Schwerdtle, T., and Walter, I. (2003) Modulation of DNA repair processes by arsenic and selenium compounds. Toxicology 193, 161-169.
(19) Zhang, X. W., Yan, X. J., Zhou, Z. R., Yang, F. F., Wu, Z. Y., Sun, H. B., Liang, W. X., Song, A. X., Lallemand-Breitenbach, V., Jeanne, M., Zhang, Q. Y., Yang, H. Y., Huang, Q. H., Zhou, G. B., Tong, J. H., Zhang, Y., Wu, J. H., Hu, H. Y., de The, H., Chen, S. J., and Chen, Z. (2010) Arsenic trioxide controls the fate of the PML-RAR oncoprotein by directly binding PML. Science 328, 240-243.
(20) Sun, X., Zhou, X., Du, L., Liu, W., Liu, Y., Hudson, L. G., and Liu, K. J. (2013) Arsenite binding-induced zinc loss from PARP-1 is equivalent to zinc deficiency in reducing PARP-1 activity, leading to inhibition of DNA repair. Toxicol. Appl. Pharmacol.
(21) Zhou, X., Sun, X., Cooper, K. L., Wang, F., Liu, K. J., and Hudson, L. G. (2011) Arsenite interacts selectively with zinc finger proteins containing C3H1 or C4 motifs. J. Biol. Chem. 286, 22855-22863.
(22) Payne, J. C., Rous, B. W., Tenderholt, A. L., and Godwin, H. A. (2003) Spectroscopic determination of the binding affinity of Zinc to the DNA-binding domains of nuclear hormone receptors. Biochemistry 42, 14214-14224.
(23) Kopera, E., Schwerdtle, T., Hartwig, A., and Bal, W. (2004) Co(II) and Cd(II) substitute for Zn(II) in the zinc finger derived from the DNA repair protein XPA, demonstrating a variety of potential mechanisms of toxicity. Chem. Res. Toxicol. 17, 1452-1458.
(24) Spuches, A. M., Kruszyna, H. G., Rich, A. M., and Wilcox, D. E. (2005) Thermodynamics of the As(III)-thiol interaction: Arsenite and Monomethylarsenite complexes with glutathione, dihydrolipoic acid, and other thiol ligands. Inorg. Chem. 44, 2964-2972.
(25) Asahina, H., Kuraoka, I., Shirakawa, M., Morita, E. H., Miura, N., Miyamoto, I., Ohtsuka, E., Okada, Y., and Tanaka, K. (1994) The XPA protein is a zinc metalloprotein with an ability to recognize various kinds of DNA damage. Mutat. Res./DNA Repair 315, 229-237.
(26) Ali, A. A. E., Timinszky, G., Arribas-Bosacoma, R., Kozlowski, M., Hassa, P. O., Hassler, M., Ladurner, A. G., Pearl, L. H., and Oliver, A. W. (2012) The zinc-finger domains of PARP1 cooperate to recognize DNA strand breaks. Nat. Struct. Mol. Biol. 19, 685-692.
(27) Fersht, A. (1999) Structure and mechanism in protein science. W H Freeman & Company.
(28) Kitchin, K. T., and Wallace, K. (2006) Dissociation of arsenite-peptide complexes: triphasic nature, rate constants, half-lives, and biological importance. J. Biochem. Mol. Toxicol. 20, 48-56.
(29) Demicheli, C., Frézard, F., Pereira, F. A., Santos, D. M., Mangrum, J. B., and Farrell, N. P. (2011) Interaction of arsenite with a zinc finger CCHC peptide: evidence for formation of an As—Zn-peptide mixed complex. J Inorg. Biochem. 105, 1753-1758.
(30) Zhao, L., Chen, S., Jia, L., Shu, S., Zhu, P., and Liu, Y. (2012) Selectivity of arsenite interaction with zinc finger proteins. Metallomics 4, 988-994.
(31) Wnek, S. M., Kuhlman, C. L., Camarillo, J. M., Medeiros, M. K., Liu, K. J., Lau, S. S., and Gandolfi, A. J. (2011) Interdependent genotoxic mechanisms of monomethylarsonous acid: role of ROS-induced DNA damage and poly(ADP-ribose) polymerase-1 inhibition in the malignant transformation of urothelial cells. Toxicol. Appl. Pharmacol. 257, 1-13.
(32) Piatek, K., Schwerdtle, T., Hartwig, A., and Bal, W. (2008) Monomethylarsonous acid destroys a tetrathiolate zinc finger much more efficiently than inorganic arsenite: mechanistic considerations and consequences for dna repair inhibition. Chem. Res. Toxicol. 21, 600-606.
(33) Bredfeldt, T. G., Jagadish, B., Eblin, K. E., Mash, E. A., and Gandolfi, A. J. (2006) Monomethylarsonous acid induces transformation of human bladder cells. Toxicol. Appl. Pharmacol. 216, 69-79.
(34) Stýblo, M., Drobná, Z., Jaspers, I., Lin, S., and Thomas, D. J. (2002) The role of biomethylation in toxicity and carcinogenicity of arsenic: a research update. Environmental Health Perspectives 110, 767-771.

1. A method of treating cancer in a patient or subject in need, the method comprising administering to said patient or subject an effective amount of:
(a) one or more elements or compounds selected from the group consisting of arsenic, an arsenite and arsenic trioxide (ATO);
(b) optionally, at least one anticancer agent (preferably, a DNA damaging agent) and
(c) further optionally, one or more additional Poly(ADP-ribose) polymerase (PARP) inhibitors, wherein said method may be carried out in combination with radiation therapy.

2. The method according to claim 1 wherein said anticancer is a DNA damaging agent.

3. The method according to claim 1 wherein said one or more elements or compounds is arsenic trioxide (ATO).

4. The method according to claim 1 wherein said DNA damaging agent is an alkylating agent, a nitrosourea, an alkyl sulfonate, an ethylenimine, a platinum drug, an antimetabolite, an anti-tumor antibiotic, a topoisomerase inhibitor, a mitotic inhibitor, a vinca alkaloids, a targeted therapy or a mixture thereof.

5. The method according to claim 1 wherein said anticancer agent is mechlorethamine (nitrogen mustard), chlorambucil, cyclophosphamide, ifosfamide, and melphalan, streptozocin, carmustine (BCNU), lomustine, busulfan, dacarbazine (DTIC), temozolomide; thiotepa, altretamine (hexamethylmelamine), cisplatin, carboplatin and oxalaplatin, fluorouracil (5-FU), 6-mercaptopurine (6-MP), capecitabine, cladribine, clofarabine, cytarabine, floxuridine, fludarabine, gemcitabine, hydroxyurea, methotrexate, pemetrexed, pentostatin, thioguanine, daunorubicin, doxorubicin, epirubicin, idarubicin, actinomycin-D, bleomycin, mitomycin-C; topotecan, irinotecan, etoposide (VP-16), teniposide, mitoxantrone, paclitaxel, docetaxel, ixabepilone, vinblastine, vincristine, vinorelbine, estramustine, imatinib, gefitinib, sunitinib, bortezomib and mixtures thereof.

6. The method according to claim 1 wherein said cancer is selected from the group consisting of carcinomas, leukemias, myeloproliferative diseases; sarcomas, tumors of the central nervous system; germ-line tumors, lung cancer, ovarian cancer, testicular cancer, thyroid cancer, astrocytoma, esophageal cancer, pancreatic cancer, stomach cancer, liver cancer, colon cancer, melanoma, carcinosarcomas benign and malignant lymphomas, benign and malignant melanomas and tumors of mixed origin.

7. The method according to claim 1 wherein said cancer is selected from the group consisting of a squamous-cell carcinomas, adenocarcinomas, hepatocellular carcinomas, renal cell carcinomas, Burkitt's lymphoma, Non-Hodgkin's lymphoma, Ewing's sarcoma, hemangiosarcoma, Kaposi's sarcoma, liposarcoma, myosarcomas, peripheral neuroepithelioma, synovial sarcoma, glioma, astrocytoma, oligodendroglioma, ependymomas, gliobastomas, neuroblastomas, ganglioneuromas, gangliogliomas, medulloblastomas, pineal cell tumors, meningiomas, meningeal sarcomas, neurofibromas, Schwannomas, Hodgkin's disease, Wilms' tumor and teratocarcinomas.

8. The method according to claim 1 wherein said cancer is a cancer of the bladder, bowel, breast, cervix, colon, esophagus, head, kidney, liver, lung, neck, ovary, pancreas, prostate, or stomach.

9. The method according to claim 1 wherein said additional PARP inhibitor is selected from the group consisting of NU1025, 3-aminobenzamide; 4-amino-1,8-naphthalimide, 1,5-isoquinolinediol, 6(5H)-phenanthriddinone, 1,3,4,5,-tetrahydrobenzo(c)(1,6)- and (c)(1,7)-naphthyridin-6 ones; adenosine substituted 2,3-dihydro-1H-isoindol-1-ones; AG14361, AG014699; 2-(4-chlorophenyl)-5-quinoxalinecarboxamide, 5-chloro-2-[3-(4-phenyl-3,6-dihydro-1 (2H)-pyridinyl)propyl]-4(3H)-quinazolinone, isoindolinone derivative INO-1001, 4-hydroxyquinazoline; 2-[3-[4-(4-chlorophenyl) 1-piperazinyl]propyl]-4-3(4)-quinazolinone, 1,5-dihydroxyisoquinoline (DHIQ); 3,4-dihydro-5 [4-(1-piperidinyl)(butoxy)-1(2H)-isoquinolone, CEP-6800, GB-15427, PJ34, DPQ, BS-201, AZD2281 (Olaparib), BS401, CHP101, CHP102, INH2BP, BSI201, BSI401, TIQ-A, an imidazobenzodiazepine, 8-hydroxy-2-methylquinazolinone (NU1025), CEP 9722, MK 4827, LT-673, 3-aminobenzamide, ABT-888 (Veliparib), BSI-201 (Iniparib), Rucaparib (AG-014699), INO-1001, A-966492, PJ-34 and mixtures thereof.

10. A method of treating a subject who suffers from a cancer selected from the group consisting of breast cancer, ovarian cancer, colorectal cancer, glioblastoma multiform (GBM), melanoma, lung cancer and a glioma, the method comprising co-administering to the subject a pharmaceutically-effective amount of:
(a) one or more elements or compounds selected from the group consisting of arsenic, an arsenite and arsenic trioxide (ATO);
(b) optionally, at least one anticancer agent (preferably, a DNA damaging agent) and
(c) further optionally, one or more additional Poly(ADP-ribose) polymerase (PARP) inhibitors, wherein said method may be carried out in combination with radiation therapy.

11. The method of claim 10, wherein the anticancer agent is a DNA damaging agent.

12. The method of claim 10 wherein the additional PARP inhibitor is selected from the group consisting of NU1025; 3-aminobenzamide; 4-amino-1,8-naphthalimide; 1,5-isoquinolinediol; 6(5H)-phenanthriddinone; 1,3,4,5,-tetrahydrobenzo(c)(1,6)- and (c)(1,7)-naphthyridin-6 ones; adenosine substituted 2,3-dihydro-1H-isoindol-1-ones; AG14361; AG014699; 2-(4-chlorophenyl)-5-quinoxalinecarboxamide; 5-chloro-2-[3-(4-phenyl-3,6-dihydro-1 (2H)-pyridinyl)propyl]-4(3H)-quinazolinone; isoindolinone derivative WO-1001; 4-hydroxyquinazoline; 2-[3-[4-(4-chlorophenyl) 1-piperazinyl]propyl]-4-3(4)-quinazolinone; 1,5-dihydroxyisoquinoline (DHIQ); 3,4-dihydro-5 [4-(1-piperidinyl)(butoxy)-1(2H)-isoquinolone; CEP-6800; GB-15427; PJ34; DPQ; BS-201; AZD2281 (Olaparib); BS401; CHP101; CHP102; INH2BP; BSI201; BSI401; TIQ-A; an imidazobenzodiazepine; 8-hydroxy-2-methylquinazolinone (NU1025), CEP 9722, MK 4827, LT-673; 3-aminobenzamide; Olaparib (AZD2281; ABT-888 (Veliparib); BSI-201 (Iniparib); Rucaparib (AG-014699); INO-1001; A-966492; PJ-34; and the PARP1 inhibitors described in U.S. patent application Ser. No. 12/576,410.

13. The method according to claim 10 wherein said one or more elements or compounds is arsesnic trioxide (ATO).

14. The method according to claim 10 wherein said anticancer agent is a DNA damaging agent selected from the group consisting of an alkylating agent, a nitrosourea, an alkyl sulfonate, an ethylenimine, a platinum drug, an antimetabolite, an anti-tumor antibiotic, a topoisomerase inhibitor, a mitotic inhibitor, a vinca alkaloids, a targeted therapy and mixtures thereof.

15. The method according to claim 10 wherein said anticancer agent is mechlorethamine (nitrogen mustard), chlorambucil, cyclophosphamide, ifosfamide, and melphalan, streptozocin, carmustine (BCNU), lomustine, busulfan, dacarbazine (DTIC), temozolomide; thiotepa, altretamine (hexamethylmelamine), cisplatin, carboplatin and oxalaplatin, fluorouracil (5-FU), 6-mercaptopurine (6-MP), capecitabine, cladribine, clofarabine, cytarabine, floxuridine, fludarabine, gemcitabine, hydroxyurea, methotrexate, pemetrexed, pentostatin, thioguanine, daunorubicin, doxorubicin, epirubicin, idarubicin, actinomycin-D, bleomycin, mitomycin-C; topotecan, irinotecan, etoposide (VP-16), teniposide, mitoxantrone, paclitaxel, docetaxel, ixabepilone, vinblastine, vincristine, vinorelbine, estramustine, imatinib, gefitinib, sunitinib, bortezomib and mixtures thereof.

16. The method of claim 1, wherein the subject is also treated by radiotherapy and the one or more elements or compounds selected from the group consisting of arsenic, an arsenite and arsenic trioxide (ATO) are administered to the subject as a radiosensitizer prior to or during radiotherapy.

17. The method of claim 10, wherein the one or more elements or compounds is ATO.

18.-62. (canceled)

63. A method of treating a subject who suffers from an inflammatory disorder, the method comprising co-administering to the subject a pharmaceutically-effective amount of:
(a) one or more elements or compounds selected from the group consisting of arsenic, an arsenite and arsenic trioxide (ATO); and optionally
(b) one or more Poly(ADP-ribose) polymerase (PARP) inhibitors.

64. The method of claim 63, wherein the PARP inhibitor is selected from the group consisting of NU1025; 3-aminobenzamide; 4-amino-1,8-naphthalimide; 1,5-isoquinolinediol; 6(5H)-phenanthriddinone; 1,3,4,5,-tetrahydrobenzo(c)(1,6)- and (c)(1,7)-naphthyridin-6 ones; adenosine substituted 2,3-dihydro-1H-isoindol-1-ones; AG14361; AG014699; 2-(4-chlorophenyl)-5-quinoxalinecarboxamide; 5-chloro-2-[3-(4-phenyl-3,6-dihydro-1 (2H)-pyridinyl)propyl]-4(3H)-quinazolinone; isoindolinone derivative INO-1001; 4-hydroxyquinazoline; 2-[3-[4-(4-chlorophenyl) 1-piperazinyl]propyl]-4-3(4)-quinazolinone; 1,5-dihydroxyisoquinoline (DHIQ); 3,4-dihydro-5 [4-(1-piperidinyl)(butoxy)-1(2H)-isoquinolone; CEP-6800; GB-15427; PJ34; DPQ; BS-201; AZD2281 (Olaparib); BS401; CHP101; CHP102; INH2BP; BSI201; BSI401; TIQ-A; an imidazobenzodiazepine; 8-hydroxy-2-methylquinazolinone (NU1025), CEP 9722, MK 4827, LT-673; 3-aminobenzamide; Olaparib (AZD2281; ABT-888 (Veliparib); BSI-201 (Iniparib); Rucaparib (AG-014699); INO-1001; A-966492; PJ-34; and the PARP1 inhibitors described in U.S. patent application Ser. No. 12/576,410.

65. The method of claim 63 wherein the one or more elements or compounds is ATO.

66. A pharmaceutical formulation which is useful in the treatment of a cancer and which comprises:
(a) one or more elements or compounds selected from the group consisting of arsenic, an arsenite and arsenic trioxide (ATO);
(b) one or more Poly(ADP-ribose) polymerase (PARP) inhibitors;
(c) optionally, one or more anticancer agents; and, optionally
(c) a pharmaceutically-acceptable excipient.

67. The composition according to claim 66 wherein said anticancer agent is at least one platinum-based anti-neoplastic agent.

68.-87. (canceled)
